SEQUENCE LISTING (44 sequences)

SEQ ID NO: 1 - protein, 7 aa, Artificial Sequence
  Description of Artificial Sequence Synthetic peptide
ANVAENA

SEQ ID NO: 2 - protein, 6 aa, Artificial Sequence
  Description of Artificial Sequence Synthetic peptide
LIAEAK

SEQ ID NO: 3 - protein, 6 aa, Artificial Sequence
  Description of Artificial Sequence Synthetic peptide
LIAPEA

SEQ ID NO: 4 - protein, 6 aa, Artificial Sequence
  Description of Artificial Sequence Synthetic peptide
  MOD_RES (2): alpha-amino isobutyric acid
LXAEAK

SEQ ID NO: 5 - protein, 6 aa, Artificial Sequence
  Description of Artificial Sequence Synthetic peptide
  MOD_RES (3): N-methyl alanine
LIXEAK

SEQ ID NO: 6 - protein, 6 aa, Artificial Sequence
  Description of Artificial Sequence Synthetic peptide
  MOD_RES (2): alpha-amino butyric acid
LXAEAK

SEQ ID NO: 7 - protein, 7 aa, Artificial Sequence
  Description of Artificial Sequence Synthetic peptide
GGQIANI

SEQ ID NO: 8 - protein, 6 aa, Artificial Sequence
  Description of Artificial Sequence Synthetic peptide
EGIAGK

SEQ ID NO: 9 - protein, 6 aa, Artificial Sequence
  Description of Artificial Sequence Synthetic peptide
LIADAK

SEQ ID NO: 10 - protein, 6 aa, Artificial Sequence
  Description of Artificial Sequence Synthetic peptide
LIANAK

SEQ ID NO: 11 - protein, 6 aa, Artificial Sequence
  Description of Artificial Sequence Synthetic peptide
LIAEAA

SEQ ID NO: 12 - protein, 6 aa, Artificial Sequence
  Description of Artificial Sequence Synthetic peptide
LIAQAK

SEQ ID NO: 13 - protein, 6 aa, Artificial Sequence
  Description of Artificial Sequence Synthetic peptide
LIAGGE

SEQ ID NO: 14 - protein, 6 aa, Artificial Sequence
  Description of Artificial Sequence Synthetic peptide
LIAGEG

SEQ ID NO: 15 - protein, 6 aa, Artificial Sequence
  Description of Artificial Sequence Synthetic peptide
ANVAEK

SEQ ID NO: 16 - protein, 6 aa, Artificial Sequence
  Description of Artificial Sequence Synthetic peptide
LIAKGK

SEQ ID NO: 17 - protein, 15 aa, Artificial Sequence
  Description of Artificial Sequence Synthetic peptide
GTPGPQGIAGQRGVV

SEQ ID NO: 18 - protein, 6 aa, Artificial Sequence
  Description of Artificial Sequence Synthetic peptide
  MOD_RES (2): alpha-amino isobutyric acid
IXAEAK

SEQ ID NO: 19 - protein, 6 aa, Artificial Sequence
  Description of Artificial Sequence Synthetic peptide
  MOD_RES (2): alpha-amino butyric acid
LXAEAK

SEQ ID NO: 20 - protein, 6 aa, Artificial Sequence
  Description of Artificial Sequence Synthetic peptide
LPAEAK

SEQ ID NO: 21 - protein, 6 aa, Artificial Sequence
  Description of Artificial Sequence Synthetic peptide
LIPEAK

SEQ ID NO: 22 - protein, 6 aa, Artificial Sequence
  Description of Artificial Sequence Synthetic peptide
  MOD_RES (3): alpha-amino isobutyric acid
LIXEAK

SEQ ID NO: 23 - protein, 6 aa, Artificial Sequence
  Description of Artificial Sequence Synthetic peptide
  MOD_RES (4): alpha-amino isobutyric acid
LIAXEA

SEQ ID NO: 24 - protein, 8 aa, Artificial Sequence
  Description of Artificial Sequence Synthetic peptide
  MOD_RES (7): b-Alanine
IWGLDGXK

SEQ ID NO: 25 - protein, 8 aa, Artificial Sequence
  Description of Artificial Sequence Synthetic peptide
  MOD_RES (7): b-Alanine
WIALEGXK

SEQ ID NO: 26 - protein, 9 aa, Artificial Sequence
  Description of Artificial Sequence Synthetic peptide
GPQGIAGQR

SEQ ID NO: 27 - protein, 6 aa, Artificial Sequence
  Description of Artificial Sequence Synthetic peptide
QGIAGQ

SEQ ID NO: 28 - protein, 7 aa, Artificial Sequence
  Description of Artificial Sequence Synthetic peptide
QGIAGQR

SEQ ID NO: 29 - protein, 6 aa, Artificial Sequence
  Description of Artificial Sequence Synthetic peptide
FGIAGF

SEQ ID NO: 30 - protein, 5 aa, Artificial Sequence
  Description of Artificial Sequence Synthetic peptide
GIAGQ

SEQ ID NO: 31 - protein, 6 aa, Artificial Sequence
  Description of Artificial Sequence Synthetic peptide
QGAIAQ

SEQ ID NO: 32 - protein, 6 aa, Artificial Sequence
  Description of Artificial Sequence Synthetic peptide
FGIAGF

SEQ ID NO: 33 - protein, 6 aa, Artificial Sequence
  Description of Artificial Sequence Synthetic peptide
CGIAGC

SEQ ID NO: 34 - protein, 6 aa, Artificial Sequence
  Description of Artificial Sequence Synthetic peptide
EGIAGK

SEQ ID NO: 35 - protein, 4 aa, Artificial Sequence
  Description of Artificial Sequence Synthetic peptide
  MOD_RES (1): variable amino acid
XIAA

SEQ ID NO: 36 - protein, 3 aa, Artificial Sequence
  Description of Artificial Sequence Synthetic peptide
  MOD_RES (3): variable amino acid
IAX

SEQ ID NO: 37 - protein, 4 aa, Artificial Sequence
  Description of Artificial Sequence Synthetic peptide
  MOD_RES (1): variable amino acid
  MOD_RES (4): variable amino acid
XIAX

SEQ ID NO: 38 - protein, 6 aa, Artificial Sequence
  Description of Artificial Sequence Synthetic peptide
  MOD_RES (3): N-methyl alanine
IIXEAK

SEQ ID NO: 39 - protein, 6 aa, Artificial Sequence
  Description of Artificial Sequence Synthetic peptide
  MOD_RES (3): D-alanine
LIXEAK

SEQ ID NO: 40 - protein, 6 aa, Artificial Sequence
  Description of Artificial Sequence Synthetic peptide
  MOD_RES (4): D-glutamic acid
LIAXAK

SEQ ID NO: 41 - protein, 6 aa, Artificial Sequence
  Description of Artificial Sequence Synthetic peptide
  MOD_RES (5): D-glutamic acid
  MOD_RES (5): variable amino acid
LIAPXA

SEQ ID NO: 42 - protein, 6 aa, Artificial Sequence
  Description of Artificial Sequence Synthetic peptide
  MOD_RES (4): trans-4-hydroxyproline
  MOD_RES (4): variable amino acid
LIAXAK

SEQ ID NO: 43 - protein, 4 aa, Artificial Sequence
  Description of Artificial Sequence Synthetic peptide
LIAE

SEQ ID NO: 44 - protein, 4 aa, Artificial Sequence
  Description of Artificial Sequence Synthetic peptide
NVAE

(19) United States
(12) Patent Application Publication
(10) Pub. No.: US 2006/0293227 A1
(43) Pub. Date: Dec. 28, 2006

(54) Cosmetic compositions and methods using transforming growth factor-beta mimics

(76) Inventor: Rajendra S. Bhatnagar, Burlingame, CA (US)

Correspondence Address: WILSON SONSINI GOODRICH & ROSATI, 650 PAGE MILL ROAD, PALO ALTO, CA, 94304-1050, US

(21) Appl. No.: 11/166,259

(22) Filed: Jun. 24, 2005

(51) Int. Cl. A61K 38/08; A61K 38/18

(52) U.S. Cl. 514/12; 514/16; 514/17

(57) ABSTRACT

The invention provides methods, compositions, and kits for a variety of cosmetic uses, where the methods, compositions and kits utilize substances that display one or more activities of transforming growth factor-β (TGF-β mimics). Compositions, methods, and kits contain TGF-β mimics.

### BACKGROUND OF THE INVENTION

Skin is subject to a number of conditions that can lead to a desire for cosmetic enhancement. Such conditions include aging due to chronological aging or photoaging from exposure to the sun, or both. Skin aging results in wrinkling, the appearance of pigmented areas (“age spots”), thinning of the skin, loss of elasticity, and other undesirable characteristics. Other skin conditions that can be improved by cosmetic approaches include scarring, e.g., from acne or other causes, uneven pigmentation, and the like. A variety of procedures have been developed for improving skin appearance. Examples of such procedures include treatment with Botulinum Toxin Type A (“botox”), retinoids and derivatives thereof and especially retinoic acid (all-trans and 13-cis) and retinol, and the use of hydroxy acids. Although progress has been made in the use of cosmetic compositions for the treatment of skin, response to treatment is variable and often a condition is only marginally to moderately responsive to treatment. Unfortunately, once applied to the skin, some of these agents can cause itching, stinging and tightness which may lead to considerable discomfort. For many subsjects, sensitivity to sun is enhanced. The use of these products by consumers with sensitive skin is often prevented. Many of the same considerations apply to other methods for cosmetic treatment of skin, such as laser resurfacing or dermabrasion. Thus, there remains a need for cosmetic and/or dermatological compositions and methods for the treatment of skin.

### SUMMARY OF THE INVENTION

The invention provides methods, compositions, and kits for the cosmetic use of TGF-β mimics.

In one aspect, the invention provides methods utilizing TGF-β mimics. In some embodiments of this aspect, the invention provides a method for cosmetic treatment of skin in an individual that includes topically administering to an individual desiring or in need of cosmetic treatment an effective amount of a TGF-β mimic. In some embodiments, the cosmetic treatment is used to modulate a cosmetic condition such as skin aging, cosmetic defect, undesired pigmentation, or post-cosmetic procedure damage. In some embodiments, the cosmetic treatment is used to augment an area of skin or epithelium (e.g., lips). In some embodiments where the cosmetic condition is a cosmetic defect, the cosmetic defect can be, e.g., striae gravidorum, striae distensiae, atrophic scarring, wound or surgical scarring, or hair loss. In some embodiments where the cosmetic condition is post-cosmetic procedure damage, the post-cosmetic procedure damage can result from, e.g., chemical peel, dermabrasion, laser resurfacing, ablative resurfacing, nonablative resurfacing, photodynamic therapy, noncoherent light phototherapy, breast lift, face lift, eyelid lift, forehead lift, neck lift, thigh lift, buttock lift, tummy tuck, and scar revision. In some embodiments, the cosmetic condition is skin aging. In some embodiments, the skin aging can result in, e.g., wrinkling, loss of elasticity, sagging, uneven pigmentation, and loss of underlying tissue mass.

In embodiments of methods of the invention, the TGF-β mimic is administered at an average frequency of about once per day to about five times per day. In some embodiments, the TGF-β mimic is administered at an average frequency of about once per day to about once per week. In some embodiments, the TGF-β mimic is administered at an average frequency of less than about once per day.

In embodiments of methods of the invention, the TGF-β mimic is applied in a vehicle such as, e.g., a spray, ointment, gel, lotion, milk, liposomal preparation, or patch. In some embodiments, the vehicle is a lotion. In some embodiments of the invention, the lotion includes a mixture of emulsifying lanolin alcohols, waxes, and oils. In some embodiments, the lotion contains water, mineral oil, isopropyl myristate, PEG40 sorbitan peroleate, glyceryl lanolate, sorbitol, propylene glycol, cetyl palmitate, magnesium sulfate, aluminum stearate, lanolin alcohol, BHT, methylchloroisothiazolinone, and methylisothiazolinone. In some embodiments, the lotion contains petrolatum or mineral oil, a quaternary ammonium, a fatty alcohol, and a fattey ester emollient. In some embodiments, the lotion contains water, glycerin, distearyldimonium chloride, petrolatum, isopropyl palmitate, cetyl alcohol, dimethicone, sodium chloride, methylparaben, and propylparaben. In some embodiments where the vehicle is a lotion, the lotion contains the TGF-β mimic at a concentration of about 0.0001% to about 0.01% by weight. In some embodiments where the vehicle is a lotion, the lotion contains the TGF-β mimic at a concentration of about 0.001% by weight.

In some embodiments of the methods of the invention, the TGF-β mimic is administered in combination with one or more other cosmetic or dermatological agents. In some embodiments, the other cosmetic or dermatological agent or agents is hydroxy acids, retinoic acid derivatives, free radical scavengers, botulinum toxin, sunscreens, anti-acne agents, and anticellulite agents.

In another aspect, the invention provides compositions. In some embodiments of this aspect, the compositions include a TGF-β mimic in a cosmetically acceptable vehicle at a concentration of greater than about 0.00005% by weight. In some embodiments of this aspect, the compositions include a TGF-β mimic at a concentration of about 0.0001% to about 0.01% by weight. In some embodiments of the invention, the vehicle includes a mixture of emulsifying lanolin alcohols, waxes, and oils. In some of the compositions of the invention, the vehicle includes water, mineral oil, isopropyl myristate, PEG-40 sorbitan peroleate, glyceryl lanolate, sorbitol, propylene glycol, cetyl palmitate, magnesium sulfate, aluminum stearate, lanolin alcohol, BHT, methylchloroisothiazolinone, methylisothiazolinone. In some embodiments, these compositions may include the TGF-β mimic at a concentration of about 0.001% by weight. In some embodiments, the vehicle contains petrolatum or mineral oil; a quaternary ammonium, a fatty alcohol, and a fattey ester emollient. In some of the compositions of the invention, the vehicle includes water, glycerin, distearyldimonium chloride, petrolatum, isopropyl palmitate, cetyl alcohol, dimethicone, sodium chloride, methylparaben, and propylparaben. In some embodiments, these compositions may include the TGF-β mimic at a concentration of about 0.001% by weight.

In yet another aspect, the invention provides kits for use in the cosmetic treatment of skin. In some embodiments of this aspect of the invention, the kit includes a composition containing a TGF-β mimic in a cosmetically acceptable vehicle and instructions for use of the composition in the cosmetic treatment of skin.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A and 1B depict the spatial coordinates for the atoms in a structure defining the biologically active TGF-β mimics useful in the methods of the invention.

### DETAILED DESCRIPTION OF THE INVENTION

The present invention provides methods, compositions, and kits for the cosmetic use of transforming growth factor-beta (TGF-β) mimics. In one aspect, the invention provides methods of cosmetic application of TGF-β mimics for cosmetic treatment of skin in an individual by topically administering an effective amount of a TGF-β mimic to the individual. In embodiments of this aspect, the cosmetic treatment is used to modulate a cosmetic condition such as skin aging (which can include wrinkling, uneven pigmentation, sagging, and the like), cosmetic defect, undesired pigmentation, post-cosmetic procedure damage, and scarring due to a skin condition. In some embodiments, the TGF-β mimic is used in combination with other cosmetic or dermatological agents or methods.

In another aspect, the invention provides compositions suitable for topical cosmetic use that include a TGF-β mimic. In some embodiments the compositions include one or more additional components besides a TGF-β mimic. In still another aspect, the invention provides kits for the cosmetic application of TGF-β) mimics to skin.

I. TGF-β Mimics

The methods and compositions of the invention relate to TGF-β mimics. A “TGF-β mimic,” as that term is used herein, includes synthetic or naturally-occurring compounds. It can be any suitable molecule, such as a peptide, peptidomimetic, peptide nucleic acid, antibody, or small or large inorganic or organic molecule, or a mixture of two or more of these types of compounds (e.g., compound that contains peptide and peptidomimetic portions), that is capable of assuming a conformation that causes it to have at least one activity of TGF-β, as measured by one or more of the assays described herein. This conformation may be a conformation that is substantially similar to or the same as the structure of a region of TGF-β, as discussed more fully below. Alternatively, this conformation may be any suitable conformation that results in an effect similar to a natural TGF-β, even if the conformation is not similar to that of a natural TGF-β.

1. Structure and Activity

Examples of TGF-β mimics are described in, e.g., U.S. Pat. Nos. 5,661,127; 5,780,436; and 6,638,912. These include compounds that are structurally or biologically analogous to a region of TGF-β and mimic the conformation recognized by TGF-β binding species (e.g., TGF-β receptors).

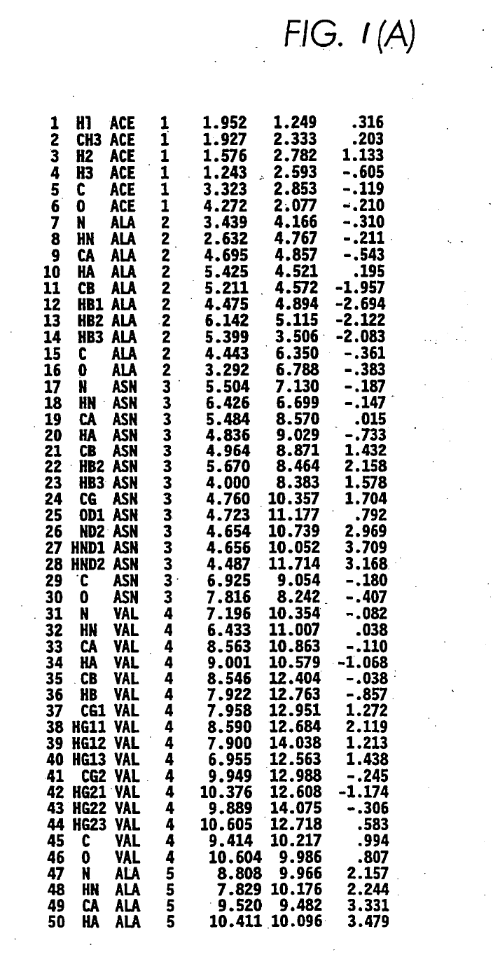

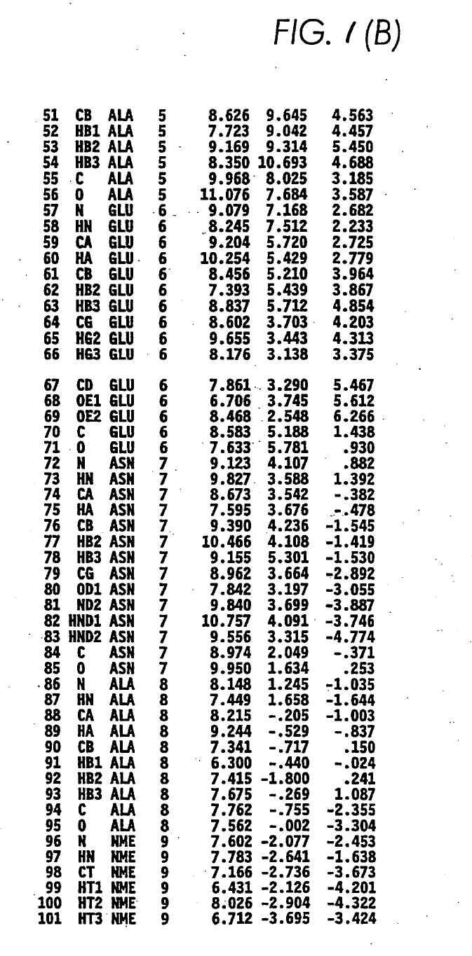

FIGS. 1A and 1B set out the coordinates for the atoms in a structure defining the biologically active TGF-β mimics useful in the methods of the invention. The coordinate computations were carried out using as a model compound, cytomodulin, described in U.S. Pat. No. 5,661,127, having the amino acid sequence Ala-Asn-Val-Ala-Glu-Asn-Ala (SEQ ID NO:1); these coordinates were obtained by use of the AMBER and MIDAS programs. Structures with substantially these same coordinates (by “substantially” is meant to include up to about a 15% variance) will generate the desired surfaces and generally display the biological activities useful in the methods of the invention; presence of biological activity can be confirmed by the assays discussed below. The allowance for an amount of some variance is due to mobility and adaptability of fit between ligand and TGF-β receptor. Thus, such structures are considered to be TGF-β mimics for purposes of this invention.

Assays that may be used to assess TGF-β activity include those that measure the ability to promote anchorage independent growth of normal fibroblasts, for example, the growth and colony formation by NRK-49 F fibroblasts in soft agar. Other assays that may be used to determine TGF-β activity include the inhibition of DNA synthesis in Mv-1-Lu mink lung epithelial cells, the induction of increased expression of type I collagen in primary cultures of neonatal human dermal fibroblasts, and/or induction of TGF-β synthesis.

Several amino acid sequences have been found to be TGF-β mimics, and are exemplary of TGF-β mimics useful in the methods provided herein. A TGF-β mimic thus may include an amino acid sequence as defined below.

The term “amino acid” as used herein means an organic compound containing both a basic amino group and an acidic carboxyl group. Included within this term are natural amino acids (e.g., generally the L-amino acids), and amino acids and imino acids which are known to occur biologically in free or combined form but usually do not occur in proteins. Included within this term also are modified and unusual amino acids, such as those disclosed in, for example, Roberts and Vellaccio (1983) The Peptides, 5: 342-429 (e.g., D-amino acids). In addition, the term “amino acid” also includes other non-naturally occurring amino acids besides the D-amino acids, which are functional equivalents of the naturally-occurring amino acids. Such non-naturally-occurring (also referred to herein as “unnatural amino acids”) amino acids include, for example, norleucine (“Nle”), norvaline (“Nva”), β-Alanine, L- or D-naphthalanine, ornithine (“Orn”), homoarginine (homoArg) and others well known in the peptide art, such as those described in M. Bodanzsky, Principles of Peptide Synthesis, 1st and 2nd revised ed., Springer-Verlag, New York, N.Y., 1984 and 1993, and Stewart and Young, Solid Phase Peptide Synthesis, 2nd ed., Pierce Chemical Co., Rockford, Ill., 1984. Amino acids and amino acid analogs can be purchased commercially (Sigma Chemical Co.; Advanced Chemtech; RSP; Bachem; or ChemImpex) or synthesized using methods known in the art.

“Natural amino acids” include, but are not limited to, alanine, arginine, asparagine, aspartic acid, cysteine, glutamic acid, glutamine, glycine, histidine, isoleucine, leucine, lysine, methionine, phenylalanine, serine, threonine, tyrosine, tyrosine, tryptophan, proline, and valine. Natural non-protein amino acids include, but are not limited to arginosuccinic acid, citrulline, cysteine sulfinic acid, 3,4-dihydroxyphenylalanine, homocysteine, homoserine, ornithine, 3-monoiodotyrosine, 3,5-diiodotryosine, 3,5,5′-triiodothyronine, and 3,3′,5,5′-tetraiodothyronine. Modified or unusual amino acids which can be used to practice the invention include, but are not limited to, D-amino acids, hydroxylysine, 4-hydroxyproline, an N-CBZ-protected amino acid, 2,4-diaminobutyric acid, homoarginine, norleucine, N-methylaminobutyric acid, naphthylalanine, phenylglycine, β-phenylproline, tert-leucine, 4-aminocyclohexylalanine, N-methyl-norleucine, 3,4-dehydroproline, N,N-dimethylaminoglycine, N-methylaminoglycine, 4-aminopiperidine-4-carboxylic acid, 6-aminocaproic acid, trans-4-(aminomethyl)-cyclohexanecarboxylic acid, 2-, 3-, and 4-(aminomethyl)-benzoic acid, 1-aminocyclopentanecarboxylic acid, 1-aminocyclopropanecarboxylic acid, and 2-benzyl-5-aminopentanoic acid.

Standard three- and one-letter abbreviations for natural amino acid residues or amino acids apply throughout the specification unless otherwise indicated.

Unnatural amino acids that fall within the scope of this invention are by way of example and without limitation: 2-aminobutanoicacid, 2-aminopentanoic acid, 2-aminohexanoic acid, 2-aminoheptanoicacid, 2-aminooctanoic acid, 2-aminononanoic acid, 2-aminodecanoic acid, 2-aminoundecanoic acid, 2-amino-3,3-dimethylbutanoic acid, 2-amino-4,4-dimethylpentanoic acid, 2-amino-3-methylhexanoic acid, 2-amino-3-methylheptanoic acid, 2-amino-3-methyloctanoic acid, 2-amino-3-methylnonanoic acid, 2-amino-4-methylhexanoic acid, 2-amino-3-ethylpentanoic acid, 2-amino-3,4-dimethylpentanoic acid, 2-amino-3,5-dimethylhexanoic acid, 2-amino-3,3-dimethylpentanoic acid, 2-amino-3-ethyl-3-methylpentanoic acid, 2-amino-3,3-diethylpentanoic acid, 2-amino-5-methylhexanoic acid, 2-amino-6-methylheptanoic, 2-amino-7-methyloctanoic, 2-amino-2-cyclopentylacetic, 2-amino-2-cylcohexylacetic acid, 2-amino-2-(1-methylcylcohexyl)acetic acid, 2-amino-2-(2-methyl-1-methylcylcohexyl)acetic acid, 2-amino-2-(3-methyl-1-methylcylcohexyl)acetic acid, 2-amino-2-(4-methyl-methylcylcohexyl)acetic acid, 2-amino-2-(1-ethylcycolhexyl)acetic acid, 2-amino-3-(cyclohexyl)propanoic acid, 2-amino-4-(cyclohexyl)butanoic acid, 2-amino-3-(1-adamantyl)propanoic acid, 2-amino-3-butenoic acid, 2-amino-3-methyl-3-butenoic acid, 2-amino-4-pentenoic acid, 2-amino-4-hexenoic acid, 2-amino-5-heptenoic acid, 2-amino-4-methyl-4-hexenoic acid, 2-amino-5-methyl-4-hexenoic acid, 2-amino-4-methy-5-hexenoic acid, 2-amino-6-heptenoic acid, 2-amino-3,3,4-trimethyl-4-pentenoic acid, 2-amino-4-chloro-4-pentenoic, 2-amino-4,4-dichloro-3-butenoic acid, 2-amino-3-(2-methylenecyclopropyl)-propanoic acid, 2-amino-2-(2-cyclopentenyl)acetic acid, 2-amino-2-(cyclohexenyl)acetic acid, 2-amino-3-(2-cyclopentenyl)propanoic acid, 2-amino-3-(3-cyclopentenyl)propanoic acid, 2-amino-3-(1-cyclohexyl)propanoic acid, 2-amino-2-(1-cyclopentenyl)acetic acid, 2-amino-2-(1-cylcohexyl)acetic acid, 2-amino-2-(1-cylcoheptenyl)acetic acid, 2-amino-2-(1-cyclooctenyl)acetic acid, 2-amino-3-(1-cycloheptenyl)propanoic acid, 2-amino-3-(1,4-cyclohexadienyl)propanoic acid, 2-amino-3-(2,5-cyclohexadienyl)propanoic acid, 2-amino-2-(7-cycloheptatrienyl)acetic acid, 2-amino-4,5-hexadienoic acid, 2-amino-3-butynoic acid, 2-amino-4-pentyoic acid, 2-amino-4-hexynoic acid, 2-amino-4-hepten-6-ynoic acid, 2-amino-3-fluoropropanoic acid, 2-amino-3,3,3-trifluoropropanoic acid, 2-amino-3-fluorobutanoic acid, 2-amino-3-fluoropentanoic acid, 2-amino-3-fluorohexanoic acid, 2-amino-3,3-difluorobutanoic acid, 2-amino-3,3-difluoro-3-phenylpropanoic acid, 2-amino-3-perfluoroethylpropanoic acid, 2-amino-3-perfluoropropylpropanoic acid, 2-amino-3-fluoro-3-methylbutanoic acid, 2-amino-5,5,5-trifluoropentanoic acid, 2-amino-3-methyl-4,4,4-trifluorobutanoic acid, 2-amino-3-trifluoromethyl-4,4,4-trifluorobutanoic acid, 2-amino-3,3,4,4,5,5-heptafluoropentanoic acid, 2-amino-3-methyl-5-fluoropentanoic acid, 2-amino-3-methyl-4-fluoropentanoic acid, 2-amino-5,5-difluorohexanoic acid, 2-amino-4-(fluoromethyl)-5-fluoropentanoic acid, 2-amino-4-trifluoromethyl-5,5,5-trifluoropentanoic acid, 2-amino-3-fluoro-3-methylbutanoic acid, 2-amino-3-fluoro-3-phenylpentanoic acid, 2-amino-2-(1-fluorocyclopentyl)acetic acid, 2-amino-2-(1-fluorocyclohexyl)acetic acid, 2-amino-3-chloropropanoic acid acid, 2-amino-3-chlorobutanoic acid acid, 2-amino-4,4-dichlorobutanoic acid acid, 2-amino4,4,4-trichlorobutanoic acid, 2-amino-3,4,4-trichlorobutanoic acid, 2-amino-6-chlorohexanoic acid, 2-amino-4-bromobutanoic acid, 2-amino-3-bromobutanoic acid, 2-amino-3-mercaptobutanoic acid, 2-amino-4-mercaptobutanoic acid, 2-amino-3-mercapto-3,3-dimethylpropanoic acid, 2-amino-3-mercapto-3-methylpentanoic acid, 2-amino-3-mercaptopentanoic acid, 2-amino-3-mercapto-4-methylpentanoic acid, 2-amino-3-methyl-4-mercaptopentanoic acid, 2-amino-5-mercapto-5-methylhexanoic acid, 2-amino-2-(1-mercaptocyclobutyl)acetic acid, 2-amino-2-(1-mercaptocyclopentyl)acetic acid, 2-amino-2-(1-mercaptocyclohexyl)acetic acid, 2-amino-5-(methylthio)pentanoic acid, 2-amino-6-(methylthio)hexanoic acid, 2-amino-4-methylthio-3-phenylbutanoic acid, 2-amino-5-ethylthio-5-methylpentanoic acid, 2-amino-5-ethylthio-3,5,5-trimethylpentanoic acid, 2-amino-5-ethylthio-5-phenylpentanoic acid, 2-amino-5-ethylthio-5-pentanoic acid, 2-amino-5-butylthio-5-methylpentanoic acid, 2-amino-5-butylthio-3,5,5-trimethylpentanoic acid, 2-amino-5-butylthio-5-phenylpentanoic acid, 2-amino-5-(butylthio)pentanoic acid, 2-amino-3-methy4-hydroselenopentanoic acid, 2-amino-4-methylselenobutanoic acid, 2-amino-4-ethylselenobutanoic acid, 2-amino-4-benzylselenobutanoic acid, 2-amino-3-methyl-4-(methylseleno)butanoic acid, 2-amino-3-(aminomethylseleno)propanoic acid, 2-amino-3-(3-aminopropylseleno)propanoic acid, 2-amino-4-methyltellurobutanoic acid, 2-amino-4-hydroxybutanoic acid, 2-amino-4-hydroxyhexanoic acid, 2-amino-3-hydroxypentanoic acid, 2-amino-3-hydroxyhexanoic acid, 2-amino-3methyl-4-hydroxybutanoic acid, 2-amino-3-hydroxy-3-methylbutanoic acid, 2-amino-6-hydroxyhexanoic acid, 2-amino-4-hydroxyhexanoic acid, 2-amino-3-hydroxy-4-methylpentanoic acid, 2-amino-3-hydroxy-3-methylpentanoic acid, 2-amino4-hydroxy-3,3-dimethylbutanoic acid, 2-amino-3-hydroxy4-methylpentanoic acid, 2-amino-3-hydroybutanedioic acid, 2-amino-3-hydroxy-3-phenyl-propanoic acid, 2-amino-3-hydroxy-3-(4-nitrophenyl)propanoic acid, 2-amino-3-hydroxy-3-(3-pyridyl)propanoic acid, 2-amino-2-(1-hydroxycyclopropyl) acetic acid, 2-amino-3-(1-hydroxycyclohexyl)propanoic acid, 2-amino-3-hydroxy-3-phenylpropanoic acid, 2-amino-3-hydroxy-3-[3-bis (2-chloroethyl)aminophenyl]propanoic acid, 2-amino-3-hydroxy-3-(3,4-dihydroxyphenyl)propanoic acid, 2-amino-3-hydroxy-3-(3,4-methylenedioxyphenyl)propanoic acid, 2-amino-4-fluoro-3-hydroxybutanoic acid, 2-amino-4,4,4-trichloro-3-hydroxybutanoic acid, 2-amino-3-hydroxy-4-hexynoic acid, 2-amino-3,4-dihydroxybutanoic acid, 2-amino-3,4,5,6-tetrahydroxyhexanoic acid, 2-amino-4,5-dihydroxy-3-methylpentanoic acid, 2-amino-5,6-dihydroxyhexanoic acid, 2-amino-5-hydroxy-4-(hydroxymethyl)pentanoic acid, 2-amino-4,5-dihydroxy-4-(hydroxymethyl)pentanoic acid, 2-amino-3-hydroxy-5-benzyloxypentanoic acid, 2-amino-3-(2-aminoethoxy)propanoic acid, 2-amino-4-(2-aminoethoxy)butanoic acid, 2-amino-4-oxobutanoic acid, 2-amino-3-oxobutanoic acid, 2-amino-4-methyl-3-oxopentanoic acid, 2-amino-3-phenyl-3-oxopropanoic acid, 2-amino-4-phenyl-3-oxobutanoic acid, 2-amino-3-methyl-4-oxopentanoic acid, 2-amino-4-oxo-4-(4-hydroxyphenyl)butanoic acid, 2-amino-4-oxo-4-(2-furyl)butanoic acid, 2-amino-4-oxo-4-(2-nitrophenyl)butanoic acid, 2-amino-4-oxo-4-(2-amino-4-chlorophenyl)butanoic acid, 2-amino-3-(4-oxo-1-cyclohexenyl)propanoic acid, 2-amino-3-(4-oxocyclohexanyl)propanoic acid, 2-amino-3-(2,5-dimethyl-3,6-dioxo-1,4-cyclohexadienyl)propanoic acid, 2-amino-3-(1-hydroxy-5-methyl-7-oxo-cyclohepta-1,3,5-trien-2-yl)propanoic acid, 2-amino-3-(1-hydroxy-7-oxo-cyclohepta-1,3,5-trien-3-yl)propanoic acid, 2-amino-3-(1-hydroxy-7-oxo-cyclohepta-1,3,5-trien-4-yl)propanoic acid, 2-amino-4-methoxy-3-butenoic acid, 2-amino-4-(2-aminoethoxy)-3-butenoic acid, 2-amino-4-(2-amino-3-hydroxypropyl)-3-butenoic acid, 2-amino-2-(4-methoxy-1,4-cyclohexadienyl)acetic acid, 2-amino-3,3-diethoxypropanoic acid, 2-amino-4,4-dimethylbutanoic acid, 2-amino-2-(2,3-epoxycyclohexyl)acetic acid, 2-amino-3-(2,3-epoxycyclohexy)propanoic acid, 2-amino-8-oxo-9,10-epoxydecanoic acid, 2-amino-propanedioic acid, 2-amino-3-methylbutanedioic acid, 2-amino-3,3-dimethylbutanedioic acid, 2-amino-4-methylpentanedioic acid, 2-amino-3-methylpentanedioic acid, 2-amino-3-phenylpentanedioic acid, 2-amino-3-hydroxypentanedioic acid, 2-amino-3-carboxypentanedioic acid, 2-amino-4-ethylpentanedioic acid, 2-amino-4-propylpentanedioic acid, 2-amino-4-isoamylpentanedioic acid, 2-amino-4-phenylpentanedioic acid, 2-amino-hexanedioic acid, 2-amino-heptanedioic acid, 2-amino-decanedioic acid, 2-amino-octanedioic acid, 2-amino-dodecanedioic acid, 2-amino-3-methylenebutanedioic acid, 2-amino-4-methylenepentanedioic acid, 2-amino-3-fluorobutanedioic acid, 2-amino-4-fluoropentanedioic acid, 2-amino-3,3-difluorobutanedioic acid, 2-amino-3-chloropentanedioic acid, 2-amino-3-hydroxybutanedioic acid, 2-amino-4-hydroxypentanedioic acid, 2-amino-4-hydroxyhexanedioic acid, 2-amino-3,4-dihydroxypentanedioic acid, 2-amino-3-(3-hydroxypropyl)butanedioic acid, 2-amino-3-(1-carboxy-4-hydroxy-2-cyclodienyl)propanoic acid, 2-amino-3-(aceto)butanedioic acid, 2-amino-3-cyanobutanedioic acid, 2-amino-3-(2-carboxy-6-oxo-6H-pyranyl)propanoic acid, 2-amino-3-carboxybutanedioic acid, 2-amino-4-carboxypentanedioic acid, 3-amido-2-amino-3-hydroxypropanoic acid, 3-amido-2-amino-3-methylpropanoic acid, 3-amido-2-amino-3-phenylpropanoic acid, 3-amido-2,3-diaminopropanoic acid, 3-amido-2-amino-3-[N-(4-hydroxyphenyl)amino]propanoic acid, 2,3-diaminopropanoic acid, 2,3-diaminobutanoic acid, 2,4-diaminobutanoic acid, 2,4-diamino-3-methylbutanoic acid, 2,4-diamino-3-phenylbutanoic acid, 2-amino-3-(methylamino)butanoic acid, 2,5-diamino-3-methylpentanoic acid, 2,7-diaminoheptanoic acid, 2,4-diaminoheptanoic acid, 2-amino-2-(2-piperidyl)acetic acid, 2-amino-2-(1-aminocyclohexyl)acetic acid, 2,3-diamino-3-phenylpropanoic acid, 2,3-diamino-3-(4-hydroxyphenyl)propanoic acid, 2,3-diamino-3-(4-methoxyphenyl)propanoic acid, 2,3-diamino-3-[4-(N,N′-dimethyamino)phenyl]propanoic acid, 2,3-diamino-3-(3,4-dimethoxyphenyl)propanoic acid, 2,3-diamino-3-(3,4-methylenedioxyphenyl)propanoic acid, 2,3-diamino-3-(4-hydroxy-3-methoxyphenyl)propanoic acid, 2,3-diamino-3-(2-phenylethyl)propanoic acid, 2,3-diamino-3-propylpropanoic acid, 2,6-diamino-4-hexenoic acid, 2,5-diamino-4-fluoropentanoic acid, 2,6-diamino-5-fluorohexanoic acid, 2,6-diamino-4-hexynoic acid, 2,6-diamino-5,5-difluorohexanoic acid, 2,6-diamino-5,5-dimethylhexanoic acid, 2,5-diamino-3-hydroxypentanoic acid, 2,6-diamino-3-hydroxyhexanoic acid, 2,5-diamino-4-hydroxypentanoic acid, 2,6-diamino-4-hydroxyhexanoic acid, 2,6-diamino-4-oxohexanoic acid, 2,7-diaminooctanedioic acid, 2,6-diamino-3-carboxyhexanoic acid, 2,5-diamino-4-carboxypentanoic acid, 2-amino-4-(2-(N,N′-diethylamino)ethyl)pentandioic acid, 2-amino-4-(N,N′-diethylamino)pentandioic acid, 2-amino-4-(N-morpholino)pentandioic acid, 2-amino-4-(N,N′-bis(2-chloroethyl)amino)pentandioic acid, 2-amino-4-(N,N′-bis(2-hydroxyethyl)amino)pentandioic acid, 2,3,5-triaminopentanoic acid, 2-amino-3-(N-(2-aminethyl)amino)propanoic acid, 2-amino-3-((2-aminoethyl)seleno)propanoic acid, 2-amino-3-[(2-aminoethyl)thio]propanoic acid, 2-amino4-aminooxybutanoic acid, 2-amino-5-hydroxyaminopentanoic acid, 2-amino-5-[N-(5-nitro-2-pyrimidinyl)amino]pentanoic acid, 2-amino-4-[(7-nitro-2,1,3-benzoxadiazol-4-yl)amino]butanoic acid, 2-amino-3-guanidinopropanoic acid, 2-amino-3-guanidinobutanoic acid, 2-amino-4-guanidobutanoic acid, 2-amino-6-guanidohexanoic acid, 2-amino-6-ureidohexanoic acid, 2-amino-3-(2-iminoimidiazolin-4-yl)propanoic acid, 2-amino-2-(2-iminohexahydropyrimidin-4-yl)acetic acid, 2-amino-3-(2-iminohexahydropyrimidiny-4-yl)propanoic acid, 2-amino4-fluoro-5-guanidopentanoic acid, 2-amino-4-hydroxy-5-guanidopentanoic acid, 2-amino-4-guanidooxybutanoic acid, 2-amino-6-amidinohexanoic acid, 2-amino-5-(N-acetimidoylamino)pentanoic acid, 1-aminocyclopropanecarboxylic acid, 1-amino4-ethylcyclpropanecarboxylic acid, 1-aminocyclopentanecarboxylic acid, 1-aminocyclopentanecarboxylic acid, 1-amino-2,2,5,5-tetramethyl-cyclohexanecarboxylic acid, 1-aminocydoheptanecarboxylic acid, 1-aminocyclononanecarboxylic acid, 2-aminoindan-2-carboxylic acid, 2-aminonorbornane-2-carboxylic acid, 2-amino-3-phenylnorbornane-2-carboxylic acid, 3-aminotetrahydrothiophene-3-carboxylic acid, 1-amino-1,3-cyclohexanedicarboxylic acid, 3-aminopyrrolidine-3-carboxylic acid, 1,4-diaminocyclohexanecarboxylic acid, 6-alkoxy-3-amino-1,2,3,4-tetrahydrocarbazole-3-carboxylic acid, 2-aminobenzobicyclo[2,2,2]octane-2-carboxylic acid, 2-aminoindan-2-carboxylic acid, 1-amino-2-(3,4-dhydroxyphenyl)cyclopropanecarboxylic acid, 5,6-dialkoxy-2-aminoindane-2-carboxylic acid, 4,5-dihydroxy-2-aminoindan-2-caroxylic acid, 5,6-dihydroxy-2-aaminotetralin-2-carboxylic acid, 2-amino-2-cyanoacetic acid, 2-amino-3-cyanopropanoic acid, 2-amino-4-cyanobutanoic acid, 2-amino-5-nitropentanoic acid, 2-amino-6-nitrohexanoic acid, 2-amino-4-aminooxybutanoic acid, 2-amino-3-(N-nitrosohydroxyamino)propanoic acid, 2-amino-3-ureidopropanoic acid, 2-amino-4-ureidobutanoic acid, 2-amino-3-phosphopropanoic acid, 2-amino-3-thiophosphopropanoic acid, 2-amino-4-methanephosphonylbutanoic acid, 2-amino-3-(trimethylsilyl)propanoic acid, 2-amino-3-(dimethyl(trimethylsilylmethylsilyl)propanoic acid, 2-amino-2-phenylacetic acid, 2-amino-2-(3-chlorophenyl)acetic acid, 2-amino-2-(4-chlorophenyl)acetic acid, 2-amino-2-(3-fluorophenyl)acetic acid, 2-amino-2-(3-methylphenyl)acetic acid, 2-amino-2-(4ofluorophenyl)acetic acid, 2-amino-2-(4-methylphenyl)acetic acid, 2-amino-2-(4-methoxyphenyl)acetic acid, 2-amino-2-(2-fluorophenyl)acetic acid, 2-amino-2-(2-methylphenyl)acetic acid, 2-amino-2-(4-choromethylphenyl)acetic acid, 2-amino-2-(4-hydroxymethylphenyl)acetic acid, 2-amino-2-[4-(methylthiomethyl)phenyl]acetic acid, 2-amino-2-(4-bromomethylphenyl)acetic acid, 2-amino-2-(4-(methoxymethy)phenyl)acetic acid, 2-amino-2-(4-((N-benzylamino)methyl)phenyl)acetic acid, 2-amino-2-(4-hydroxylphenyl)acetic acid, 2-amino-2-(3-hydroxylphenyl)acetic acid, 2-amino-2-(3-carboxyphenyl)acetic acid, 2-amino-2-(4-aminophenyl)acetic acid, 2-amino-2-(4-azidophenyl)acetic acid, 2-amino-2-(3-t-butyl-4-hydroxyphenyl)acetic acid, 2-amino-2-(3,5-difluoro-4-hydroxyphenyl)acetic acid, 2-amino-2-(3,5-dihydroxyphenyl)acetic acid, 2-amino-2-(3-carboxy-4-hydroxyphenyl)acetic acid, 2-amino-2-(3,5-di-t-butyl-4-hydroxyphenyl)acetic acid, 2-amino-3-(2-methylphenyl)propanoic acid, 2-amino-3-(4-ethylphenyl)propanoic acid, 2-amino-3-(4-phenylphenyl)propanoic acid, 2-amino-3-(4-benzylphenyl)propanoic acid, 2-amino-3-(3-fluorophenyl)propanoic acid, 2-amino-3-(4-methylphenyl)propanoic acid, 2-amino-3-(4-fluorophenyl)propanoic acid, 2-amino-3-(4-chlorophenyl)propanoic acid, 2-amino-3-(2-chlorophenyl)propanoic acid, 2-amino-3-(4-bromophenyl)propanoic acid, 2-amino-3-(2-bromophenyl)propanoic acid, 2-amino-3-(3-hydroxyphenyl)propanoic acid, 2-amino-3-(2-hydroxyphenyl)propanoic acid, 2-amino-3-(4-mercaptophenyl)propanoic acid, 2-amino-3-(3-trifluoromethylphenyl)propanoic acid, 2-amino-3-(3-hydroxyphenyl)propanoic acid, 2-amino-3-(4-hydroxyphenyl)propanoic acid, 2-amino-3-[4-(hydroxymethy)phenyl]propanoic acid, 2-amino-3-[3-(hydroxymethyl)phenyl]propanoic acid, 2-amino-3-[3-(aminomethyl)phenyl]propanoic acid, 2-amino-3-(3-carboxyphenyl) propanoic acid, 2-amino-3-(4-nitrophenyl)propanoic acid, 2-amino-3-(4-aminophenyl) propanoic acid, 2-amino-3-(4-azidophenyl)propanoic acid, 2-amino-3-(4-cyanophenyl)propanoic acid, 2-amino-3-(4-acetophenyl)propanoic acid, 2-amino-3-(4-guanidinophenyl)propanoic acid, 2-amino-3-[4-(phenylazo)phenyl]propanoic acid, 2-amino-3-[4-(2-phenylethylenyl)phenyl]propanoic acid, 2-amino-3-(4-trialkylsilylphenyl)propanoic acid, 2-amino-3-(2,4-dimethylphenyl)propanoic acid, 2-amino-3-(2,3-dimethylphenyl)propanoic acid, 2-amino-3-(2,5-dimethylphenyl) propanoic acid, 2-amino-3-(3,5-dimethylphenyl)propanoic acid, 2-amino-3-(2,4,6-trimethylphenyl)propanoic acid, 2-amino-3-(3,4,5-trimethylphenyl)propanoic acid, 2-amino-3-(2,3,4,5,6-pentamethylphenyl)propanoic acid, 2-amino-3-(2,4,-difluorophenyl)propanoic acid, 2-amino-3-(3,4,6-difluorophenyl)propanoic acid, 2-amino-3-(2,5,difluorophenyl)propanoic acid, 2-amino-3-(2,6,-difluorophenyl)propanoic acid, 2-amino-3-(2,3,5,6-tetrafluorophenyl)propanoic acid, 2-amino-3-(3,5-dichloro-2,4,6-trifluorophenyl)propanoic acid, 2-amino-3-(2,3-difluorophenyl)propanoic acid, 2-amino-3-(2,3-bistrifluoromethylphenyl)propanoic acid, 2-amino-3-(2,4-bistrifluoromethylphenyl)propanoic acid, 2-amino-3-(2-chloro-5-trifluoromethylphenyl)propanoic acid, 2-amino-3-(2,5-difluorophenyl)propanoic acid, 2-amino-3-(2,3,4,5,6-pentafluorophenyl)propanoic acid, 2-amino-3-(2,3-dibromophenyl)propanoic acid, 2-amino-3-(2,5-dibromophenyl)propanoic acid, 2-amino-3-(3,4-dibromophenyl)propanoic acid, 2-amino-3-(3,4,5-triiodophenyl)propanoic acid, 2-amino-3-(2,3-dihydroxyphenyl)propanoic acid, 2-amino-3-(2,5-dihydroxyphenyl)propanoic acid, 2-amino-3-(2,6-dihydroxyphenyl)propanoic acid, 2-amino-3-(3-bromo-5-methoxyphenyl)propanoic acid, 2-amino-3-(2,5-dimethoxyphenyl)propanoic acid, 2-amino-3-(2,5-dimethoxy-4-methylphenyl)propanoic acid, 2-amino-3-(4-bromo-2,5-dimethoxyphenyl)propanoic acid, 2-amino-3-(3-carboxy-4-hydroxyphenyl)propanoic acid, 2-amino-3-(3-carboxy-4-aminophenyl)propanoic acid, 2-amino-3-(2-hydroxy-5-nitrophenyl)propanoic acid, 2-amino-3-(2-ethoxy-5-nitrophenyl)propanoic acid, 2-amino-3-(3,4,5-trimethoxyphenyl)propanoic acid, 2-amino-3-(4-azido-2-nitrophenyl)propanoic acid, 2-amino-3-(2-hydroxy-5-nitrophenyl)propanoic acid, 2-amino-3-(2,4-bis-trimethylsilylphenyl)propanoic acid, 2-amino-3-(4-hydroxy-3,5-di-t-butylphenyl)propanoic acid, 2-amino-3-(4-hydroxy-3-benzylphenyl)propanoic acid, 2-amino-3-(4-hydroxy-3-fluorophenyl)propanoic acid, 2-amino-3-(4-hydroxy-2,3,5,6-tetrafluorophenyl)propanoic acid, 2-amino-3-(4-hydroxy-3,5-dichlorophenyl)propanoic acid, 2-amino-3-(4-hydroxy-3-iodophenyl)propanoic acid, 2-amino-3-(4-hydroxy-3,5-diiodophenyl)propanoic acid, 2-amino-3-(4-hydroxy-2-hydroxyphenyl)propanoic acid, 2-amino-3-(4-hydroxy-3-hydroxymethylphenyl)propanoic acid, 2-amino-3-(4-hydroxy-2hydroxy-6-methylphenyl)propanoic acid, 2-amino-3-(4-hydroxy-3-carboxyphenyl)propanoic acid, 2-amino-3-(4-hydroxy-3,5-dinitrophenyl)propanoic acid, substituted thyronines, 2-amino-3-(3,4-dihydroxy-2-chlorophenyl)propanoic acid, 2-amino-3-(3,4-dihydroxy-2-bromophenyl)propanoic acid, 2-amino-3-(3,4-dihydroxy-2-fluorophenyl)propanoic acid, 2-amino-3-(3,4-dihydroxy-2-nitrophenyl)propanoic acid, 2-amino-3-(3,4-dihydroxy-2-methylphenyl)propanoic acid, 2-amino-3-(3,4-dihydroxy-2-ethylphenyl)propanoic acid, 2-amino-3-(3,4-dihydroxy-2-isopropylphenyl)propanoic acid, 2-amino-3-(2-t-butyl-4,5-dihydroxyphenyl)propanoic acid, 2-amino-3-(3-fluoro-4,5-dihydroxyphenyl)propanoic acid, 2-amino-3-(2-fluoro-4,5-dihydroxyphenyl)propanoic acid, 2-amino-3-(2,5,6-trifluoro-3,4-dihydroxyphenyl)propanoic acid, 2-amino-3-(2,6-dibromo-3,4-dihydroxyphenyl)propanoic acid, 2-amino-3-(5,6-dibromo-3,4-dihydroxyphenyl)propanoic acid, 2-amino-3-(2,4,5-trihydroxyphenyl)propanoic acid, 2-amino-3-(2,3,4-trihydroxyphenyl)propanoic acid, 2-amino-3-(3,4-dihydroxy-5-methoxyphenyl)propanoic acid, 2-amino-3-methyl-3-phenylpropanoic acid, 2-amino-3-ethyl-3-phenylpropanoic acid, 2-amino-3-isopropyl-3-phenylpropanoic acid, 2-amino-3-butyl-3-phenylpropanoic acid, 2-amino-3-benzyl-3-phenylpropanoic acid, 2-amino-3-phenylethyl-3-phenylpropanoic acid, 2-amino-3-(4-chorophenyl)-3-phenylpropanoic acid, 2-amino-3-(4-methoxyphenyl)-3-phenylpropanoic acid, 2-amino-3,3-diphenylpropanoic acid, 2-amino-3-[4-(N,N-diethylamino)phenyl]heptanoic acid, 2-amino-3-[4-(N,N-diethylamino)phenyl]pentanoic acid, 2-amino-3-(3,4-dimethoxyphenyl)pentanoic acid, 2-amino-3-(3,4-dihydroxyphenyl)pentanoic acid, 2-amino-3-methyl-3-phenylbutanoic acid, 2-amino-3-ethyl-3-phenylpentanoic acid, 2-amino-3-methyl-3-phenylpentanoic acid, 2-amino-3,3-diphenylbutanoic acid, 2-amino-3-fluoro-3-phenylpropanoic acid, 2-amino-3-methylene-3-phenylpropanoic acid, 2-amino-3-methylmercapto-3-phenylpropanoic acid, 2-amino-4-methylmercapto-4-phenylbutanoic acid, 2-amino-4-(3,4-dihydroxyphenyl)butanoic acid, 2-amino-5-(4-methoxyphenyl)pentanoic acid, 2-amino-4-phenylbutanoic acid, 2-amino-5-phenylpentanoic acid, 2-amino-3,3-dimethyl-5-phenylpentanoic acid, 2-amino-4-phenyl-3-butenoic acid, 2-amino-4-phenoxybutanoic acid, 2-amino-5-phenoxypentanoic acid, 2-amino-2-(indanyl)acetic acid, 2-amino-2-(1-tetralyl)acetic acid, 2-amino-4,4-diphenylbutanoic acid, 2-amino-2-(2-naphthyl)acetic acid, 2-amino-3-(1-naphthyl)propanoic acid, 2-amino-3-(1-naphthyl)pentanoic acid, 2-amino-3-(2-naphthyl)propanoic acid, 2-amino-3-(1-chloro-2-naphthyl)propanoic acid, 2-amino-3-(1-bromo-2-naphthyl)propanoic acid, 2-amino-3-(4-hydroxy-1-naphthyl)propanoic acid, 2-amino-3-(4-methoxy-1-naphthyl)propanoic acid, 2-amino-3-(4-hydroxy-2-chloro-1-naphthyl)propanoic acid, 2-amino-3-(2-chloro-4-methoxy-1-naphthyl)propanoic acid, 2-amino-2-(2-anthryl)acetic acid, 2-amino-3-(9-anthryl)propanoic acid, 2-amino-3-(2-fluorenyl)propanoic acid, 2-amino-3-(4-fluorenyl)propanoic acid, 2-amino-3-(carboranyl)propanoic acid, 3-methylproline, 4-methylproline, 5-methylproline, 4,4-dimethylproline, 4-fluoroproline, 4,4-difluoroproline, 4-bromoproline, 4-chloroproline, 4-aminoproline, 3,4-dehydroproline, 4-methylproline, 4-methyleneproline, 4-mercaptoproline, 4-(4-methoxybenzylmercapto)proline, 4-hydroxymethylproline, 3-hydroxyproline, 3-hydroxy-5-methylproline, 3,4-dihydroxyproline, 3-phenoxyproline, 2-aminoproline, 5-aminoproline, 3-carbamylalkylproline, 4-cyano-5-methyl-5-carboxyproline, 4,5-dicarboxyl-5-methylproline, 2-aziridinecarboxylic acid, 2-azetidinecarboxylic acid, 4-methyl-2-azetidinecarboxylic acid, pipecolic acid, 1,2,3,6-tetrahydropicolinic acid, 3,4-methyleneproline, 2,4-methyleneproline, 4-aminopipecolic acid, 5-hydroxypipecolic acid, 4,5-dihydroxypipecolic acid, 5,6-dihydroxy-2,3-dihydroindole-2-carboxylic acid, 1,2,3,4-tetrahydroquinoline-2-carboxylic acid, 6,7-dihydroxy-1,2,3,4-tetrahydroisoquinoline-3-carboxylic acid, 6-hydroxy-1-methyl-1,2,3,4-tetrahydroisoquinoline-3-carboxylic acid, 6,7-dihydroxy-1-methyl-1,2,3,4-tetrahydroisoquinoline-3-carboxylic acid, 1,3-oxazolidine-4-carboxylic acid, 1,2-oxazolidine-3-carboxylic acid, perhydro-1,4-thiazine-3-carboxylic acid, 2,2-dimethylthiazolidine-4-carboxylic acid, perhydro-1,3-thiazine-2-carboxylic acid, selenazolidine4-carboxylic acid, 2-phenylthiazolidine4-carboxylic acid, 2-(4-carboxylicyl)thiazolidine-4-carboxylic acid, 1,2,3,4,4a,9a-hexahydro-beta-carboline-3-carboxylic acid, 2,3,3a,8-atetrahydropyrrolo(2,3b)indole-2-carboxylic acid, 2-amino-3-(2-pyridyl)propanoic acid, 2-amino-3-(3-pyridyl)propanoic acid, 2-amino-3-(4-pyridyl)propanoic acid, 2-amino-3-(2-bromo-3-pyridyl)propanoic acid, 2-amino-3-(2-bromo-4-pyridyl)propanoic acid, 2-amino-3-(2-bromo-5-pyridyl)propanoic acid, 2-amino-3-(2-bromo-6-pyridyl)propanoic acid, 2-amino-3-(2-chloro-3-pyridyl)propanoic acid, 2-amino-3-(2-chloro-4-pyridyl)propanoic acid, 2-amino-3-(2-chloro-5-pyridyl)propanoic acid, 2-amino-3-(2-chloro-6-pyridyl)propanoic acid, 2-amino-3-(2-fluoro-3-pyridyl)propanoic acid, 2-amino-3-(2-fluoro-4-pyridyl)loropanoic acid, 2-amino-3-(2-fluoro-5-pyridyl)propanoic acid, 2-amino-3-(2-fluoro-6-pyridyl)proloanoic acid, 2-amino-3-(1,2-dihydro-2-oxo-3-pyridyl)propanoic acid, 2-amino-3-(1,2-dihydro-2-oxo-4-pyridyl)propanoic acid, 2-amino-3-(1,2-dihydro-2-oxo-5-pyridyl)propanoic acid, 2-amino-3-(1,2-dihydro-2-oxo-6-pyridyl)propanoic acid, 2-amino-3-(5-hydroxy-2-pyridyl)propanoic acid, 2-amino-3-(5-hydroxy-6-iodo-2-pyridyl)propanoic acid, 2-amino-3-(3-hydroxy-4-oxo-1,4dihydro-1-pyridyl)propanoic acid, N-(5-caroxyl-5-aminopentyl)pyridinium chloride, 1,2,5-trimethyl-4-(2-amino-2-carboxy-1-hydroxyethyl)pyridinium chloride, 2-amino-2-(5-chloro-2-pyridyl)acetic acid, N-(3-amino-3-carboxypropyl)pyridinium chloride, 2-amino-3-(2-pyrryl)propanoic acid, 2-amino-3-(1-pyrryl)propanoic acid, 2-amino-4-(1-pyrryl)butanoic acid, 2-amino-5-(1-pyrryl)pentanoic acid, 2-amino-3-(5-imidazolyl)-3-methylpropanoic acid, 2-amino-3-(5-imidazolyl)-3-ethylpropanoic acid, 2-amino-3-hexyl-3-(5-imidazolyl)propanoic acid, 2-amino-3-hydroxy-3-(5-imidazolyl)propanoic acid, 2-amino-3-(4-nitro-5-imidazolyl)proloanoic acid, 2-amino-3-(4-methyl-5-imidazolyl)propanoic acid, 2-amino-3-(2-methyl-5-imidazolyl)propanoic acid, 2-amino-3-(4-fluoro-5-imidazolyl)propanoic acid, 2-amino-3-(2-fluoro-5-imidazolyl)propanoic acid, 2-amino-3-(2-amino-5-imidazolyl)propanoic acid, 2-amino-3-(2-phenylaza-5-imidazolyl)propanoic acid, 2-amino-3-(1-methyl-2-nitro-5-imidazolyl)propanoic acid, 2-amino-3-(1-methyl-4-nitro-5-imidazolyl)propanoic acid, 2-amino-3-(1-methyl-5-nitro-5-imidazolyl)propanoic acid, 2-amino-3-(2-mercapto-5-imidazolyl)propanoic acid, 2-amino-4-(5-imidazolyl)butanoic acid, 2-amino-3-(1-imidazolyl)propanoic acid, 2-amino-3-(2-imidazolyl)propanoic acid, 2-amino-(1-pyrazolyl)propanoic acid, 2-amino-(3-pyrazolyl)propanoic acid, 2-amino-(3,5-dialkyl-4-pyrazolyl)propanoic acid, 2-amino-3-(3-amino-1,2,4-triazol-1-yl)propanoic acid, 2-amino-3-(tetrazol-5-yl)propanoic acid, 2-amino-4-(5-tetrazolyl)butanoic acid, 2-amino-3-(6-methyl-3-indolyl)propanoic acid, 2-amino-3-(4-fluoro-3-indolyl)propanoic acid, 2-amino-3-(5-fluoro-3-indolyl)propanoic acid, 2-amino-3-(6-fluoro-3-indolyl)propanoic acid, 2-amino-3-(4,6,6,7-tetrafluoro-3-indolyl)propanoic acid, 2-amino-3-(-chloro-3-indolyl)propanoic acid, 2-amino-3-(6-chloro-3-indolyl)propanoic acid, 2-amino-3-(7-chloro-3-indolyl)propanoic acid, 2-amino-3-(6-bromo-3-indolyl)propanoic acid, 2-amino-3-(7-bromo-3-indolyl)propanoic acid, 2-amino-3-(2-hydroxy-3-indolyl)propanoic acid, 2-amino-3-(7-hydroxy-3-indolyl)propanoic acid, 2-amino-3-(7-hydroxy-3-indolyl)propanoic acid, 2-amino-3-(2-alkylmercapto-3-indolyl)propanoic acid, 2-amino-3-(7-amino-3-indolyl)propanoic acid, 2-amino-3-(4-nitro-3-indolyl)propanoic acid, 2-amino-3-(7-nitro-3-indolyl)propanoic acid, 2-amino-3-(4-carboxy-3-indolyl)propanoic acid, 2-amino-3-(3-indolyl)butanoic acid, 2-amino-3-(2,3-dihydro-3-indolyl)propanoic acid, 2-amino-3-(2,3-dihydro-2-oxo-3-indolyl)propanoic acid, 2-amino-3-alkylmercapto-3-(3-indolyl)propanoic acid, 2-amino-3-(4-aza-3-indolyl)propanoic acid, 2-amino-3-(7-aza-3-indolyl)propanoic acid, 2-amino-3-(7-aza-6-chloro-4-methyl-3-indolyl)propanoic acid, 2-amino-3-(2,3-dihydrobenzofuran-3-yl)propanoic acid, 2-amino-3-(3-methyl-5-7-dialkylbenzofuran-2-yl)propanoic acid, 2-amino-3-(benzothiophen-3-yl)propanoic acid, 2-amino-3-(5-hydroxybenzothiophen-3-yl)propanoic acid, 2-amino-3-eoenzoselenol-3yl)propanoic acid, 2-amino-3-quinolylpropanoic acid, 2-amino-3-(8-hydroxy-5-quinolyl)propanoic acid, 2-amino-2-(5,6,7,8-tetrahydroquinol-5-yl)acetic acid, 2-amino-3-(3-coumarinyl)propanoic acid, 2-amino-2-(benzisoxazol-3-yl)acetic acid, 2-amino-2-(5-methylbenzisoxazol-3-yl)acetic acid, 2-amino-2-(6-methylbenzisoxazol-3-yl)acetic acid; 2-amino-2-(7-methylbenzisoxazol-3-yl)acetic acid, 2-amino-2-(5-bromobenzisoxazol-3-yl)acetic acid, 2-amino-3-(benzimidazol-2-yl)propanoic acid, 2-amino-3-(5,6-dichlorobenzimidazol-2-yl)propanoic acid, 2-amino-3-(5,6-dimethylbenzimidazol-2-yl)propanoic acid, 2-amino-3-(4,5,6,7-hydrobenzirnidazol-2-yl)propanoic acid, 2-amino-2-(benzimidazol-5-yl)acetic acid, 2-amino-2-(1,3-dihydro-2,2-dioxoisobenzothiophen-5-yl)acetic acid, 2-amino-2-(1,3-dihydro-2,2-dioxo-2,1,3-benzothiadiazol-5-yl)acetic acid, 2-amino-2-(2-oxobenzimidazol-5-yl)acetic acid, 2-amino-3-(4-hydroxybenzothiazol-6-yl)propanoic acid, 2-amino-3-(benzoxazol-2-yl)propanoic acid, 2-amino-3-(benzothiazol-2-yl)propanoic acid, 2-amino-3-(9-adeninyl)propanoic acid, 2-amino-2-(6-chloro-9-purinyl)acetic acid, 2-amino-2-(6-amino-9-purinyl)acetic acid, 2-amino-3-(6-purinyl)propanoic acid, 2-amino-3-(8-theobrominyl)propanoic acid, 2-amino-2-(1-uracilyl)acetic acid, 2-amino-2-(1-cytosinyl)acetic acid, 2-amino-3-(1-uracilyl)propanoic acid, 2-amino-3-(1-cytosinyl)propanoic acid, 2-amino-4-(1-pyrimidinyl)butanoic acid, 2-amino-4-(4-amino-1-pyrimidinyl)butanoic acid, 2-amino-4-(4-hydroxy-1-pyrimidinyl)butanoic acid, 2-amino-5-(1-pyrimidinyl)pentanoic acid, 2-amino-5-(4-amino-1-pyrimidinyl)pentanoic acid, 2-amino-5-(4-hydroxy-1-pyrimidinyl)pentanoic acid, 2-amino-3-(5-pyrimidinyl)propanoic acid, 2-amino-3-(6-uracilyl)propanoic acid, 2-amino-3-(2-pyrimidinyl)propanoic acid, 2-amino-3-(6-amino-4-chloro-2-pyrimidinyl)propanoic acid, 2-amino-3-(4-hydroxy-2-pyrimidinyl)propanoic acid, 2-amino-3-(2-amino-4-pyrimidinyl)propanoic acid, 2-amino-3-(4,5-dihydroxypyrimidin-2-yl)propanoic acid, 2-amino-3-(2-thiouracil-6-yl)propanoic acid, 2-amino-2-(5-alkyl-2-tetrahydrofuryl)acetic acid, 2-amino-2-(5-methyl-2,5-dihydro-2-furyl)acetic acid, 2-amino-2-(5-alkyl-2-furyl)acetic acid, 2-amino-2-(2-furyl)acetic acid, 2-amino-2-(3-hydroxy-5-methyl-4-isoxazolyl)acetic acid, 2-amino-3-(4-bromo-3-hydroxy-5-isoxazolyl)propanoic acid, 2-amino-3-(4-methyl-3-hydroxy-5-isoxazolyl)propanoic acid, 2-amino-3-(3-hydroxy-5-isoxazolyl)propanoic acid, 2-amino-2-(3-chloro-D2-isoxazolin-5-yl)acetic acid, 2-amino-2-(3-oxo-5-isoxazolidinyl)acetic acid, 2-amino-3-(3,5-dioxo-1,2,4-oxadiazolin-2-yl)propanoic acid, 2-amino-3-(3-phenyl-5-isoxazolyl)propanoic acid, 2-amino-3-[3-(4-hydroxyphenyl)-1,2,4-oxadiazol-5-yl]propanoic acid, 2-amino-3-(2-thienyl)propanoic acid, 2-amino-2-(2-furyl)acetic acid, 2-amino-2-(2-thienyl)acetic acid, 2-amino-2-(2-thiazolyl)acetic acid, 2-amino-3-(2-thiazolyl)propanoic acid, 2-amino-4-(4-carboxy-2-thiazolyl)butanoic acid, 2-amino-3-(4-thiazolyl)propanoic acid, 2-amino-3-(2-selenolyl)propanoic acid, 2-amino-3-(2-amino-4-selenolyl)propanoic acid, and 2-amino-3-(beta-ribofuranosyl)propanoic acid.

“Amino acids residue” has its customary meaning in the art and refers to an amino acid that is part of a peptide or polypeptide chain; “amino acid residue” as used herein also refers to various amino acids where sidechain functional groups are coupled with appropriate protecting groups known to those skilled in the art. “The Peptides”, Vol 3, 3-88 (1981) discloses numerous suitable protecting groups. Examples of amino acids where sidechain functional groups are coupled with appropriate protecting groups include, but are not limited to, Asp(OMe), Glu(OMe), Hyp(OMe), Asp(O′Bu), Glu(O′Bu), Hyp(O′Bu), Thr(O′Bu), Asp(OBzl), Glu(OBzl), Hyp(OBzl), and Thr(OBzl).

Many TGF-β mimics are composed of, or include, a peptide. The term “peptide” as used herein refers to polymers of amino acids under 100 amino acids in length; in preferred embodiments, less than 30 amino acids in length. A peptide may be more than about 1, 2, 3, 4, 5, 6, 7, 8, 9, or 10 amino acids in length. A peptide may be less than 100, 90, 80, 70, 60, 50, 40, 35, 30, 25, 20, 15, 14, 13, 12, 11, 10, 9, 8, 7, 6, 5, 4, or 3 amino acids in length. Preferred peptides are less than 30 amino acids in length. The polymer may be linear or branched, it may comprise amino acids of any type as defined above, and it may be interrupted by non-amino acids (e.g., peptidomimetics). The term also encompasses an amino acid polymer that has been modified naturally or by intervention; for example, disulfide bond formation, glycosylation, lipidation, acetylation, carboxylation, phosphorylation, ubiquitination, pegylation or any suitable other manipulation or modification, such as conjugation with a labeling component.

The hydrophobicity, charge, ability to form hydrogen bonds, and other properties of amino acids can be involved in producing peptides that act as TGF-β mimics. Of the twenty common amino acids, those with hydrophobic R groups include: Alanine, Valine, Leucine, Isoleucine, Proline, Phenylalanine, Tryptophan and Methionine. Amino acids with uncharged polar R groups include: Glycine, Serine, Threonine, Cysteine, Tyrosine, Asparagine and Glutamine. Amino acids with charged polar R groups include: Aspartic acid and Glutamic acid. Basic amino acids include: Lysine, Arginine and Histidine (at pH 6.0). Amino acids with phenyl groups include: Phenylalanine, Tryptophan and Tyrosine. As will be apparent to those of skill in the art, many unnatural or artificial amino acids also fall within the various types of amino acids, and may be used in embodiments of the invention.

In exemplary peptides useful in the invention, the appropriate functional groups are represented, in many cases, within the following amino acid sequence, which may be the entire TGF-β mimic or may be included in a larger structure: AAᵢ-AAᵢ₊₁-AAᵢ₊₂ . . . -AAᵢ₊ₙ. In some embodiments, TGF-β mimics useful in the methods of the invention contain:
(1) a hydrophobic or neutral amino acid at position i;
(2) a branched hydrophobic at position i+1 (e.g., Val, Ile);
(3) a small aliphatic at position i+2 (e.g., Ala), where positions i+1 and i+2 together form a structure found in TGF-β mimics, the U-bend (β-bend) structure; and
(4) a side-chain containing a hydrogen-bond acceptor shortly thereafter (at position AAᵢ₊ₙ).

For example, in some embodiments, AAᵢ is alanine, asparagine, or leucine, AAᵢ₊₁ is valine or isoleucine, and AAᵢ₊₂ is alanine. One aspect of TGF-β mimics useful in the invention is the relative positioning of the side-chains of AAᵢ₊₁ and AAᵢ₊₂. The correct positioning of these amino acids can be achieved if AAᵢ₊₁ and AAᵢ₊₂ are in either of two backbone conformations: a β-bend or an “extended-bend” conformation with the backbone (Φ,Ψ) angles of AAᵢ₊₁ equal to approximately (−60, +135) and those of AAᵢ₊₂ equal to approximately (−60, −45). This sequence is often either immediately followed by or has proximal thereto an amino acid with a hydrogen bond acceptor-containing side-chain. Because the amino acid residue with a hydrogen bond acceptor-containing side-chain does not necessarily have to be immediately followed by, that is adjacent to AAᵢ₊₂, it is referred to as AAᵢ₊ₙ where n is an integer equal to or greater than three. If n is greater than 3, then n−3 (“n minus 3”) amino acid residues would be between AAᵢ₊₂ and AAᵢ₊ₙ in the peptide sequence.

For example, the TGF-β mimics may include the sequence AAᵢ-AAᵢ₊₁-AAᵢ₊₂-AAᵢ₊₃ where AAᵢ through AAᵢ₊₂ are as before described and AAᵢ₊₂ is an amino acid residue with a hydrogen bond acceptor-containing side-chain (and is glutamic acid, aspartic acid, glutamine, or asparagine). The original peptide with TGF-β activity and an especially preferred TGF-β mimic for use in the methods of the present invention, cytomodulin, A-N-V-A-E-N-A (SEQ ID NO: 1) is of this class. Here, AAᵢ-AAᵢ₊₁-AAᵢ₊₂-AAᵢ₊₃ corresponds to the second through fifth residues from the N-terminus of cytomodulin, -N-V-A-E-. Other preferred embodiments are the peptides, L-I-A-E-A-K (SEQ ID NO:2) and L-I-A-E-A-A (SEQ ID NO: 11). In these examples, AAᵢ-AAᵢ₊₁-AAᵢ₊₂-AAᵢ₊₃ corresponds to the first four residues of the peptide, L-I-A-E-.

Another example is the sequence AAᵢ-AAᵢ₊₁-AAᵢ₊₂-AAᵢ₊₃-AAᵢ₊₄ where the residue with the hydrogen bond acceptor-containing side-chain is not immediately adjacent, but instead is proximal to, the initial sequence. In this case, AAᵢ through AAᵢ₊₂ are as before, AAᵢ₊₃ is any suitable amino acid, and AAᵢ₊₄ may be glutamic acid, aspartic acid, glutamine, or asparagine. A preferred embodiment of this class is L-I-A-G-E-G (SEQ ID NO: 14). An especially preferred embodiment is the peptide, L-I-A-P-E-A (SEQ ID NO:3). In both examples, the first five N-terminal amino acids correspond to AAᵢ-AAᵢ₊₁-AAᵢ₊₂-AAᵢ₊₃-AAᵢ₊₄.

Yet another example is the sequence AAᵢ-AAᵢ₊₁-AAᵢ₊₂-AAᵢ₊₃-AAᵢ₊₄-AAᵢ₊₅. Here, AAᵢ through AAᵢ₊₂ are as before, AAᵢ₊₃ and AAᵢ₊₄ are suitable amino acids, and AAᵢ₊₅ may be glutamic acid, aspartic acid, glutamine, or asparagine. An especially preferred member of this series is the peptide, L-I-A-G-G-E (SEQ ID NO: 13). In this particular example there is a one to one correspondence between SEQ ID NO:3 and the sequence AAᵢ-AAᵢ₊₁-AAᵢ₊₂-AAᵢ₊₃-AAᵢ₊₄-AAᵢ₊₅.

As discussed above, the original peptide discovered to have TGF-β activity has been named “cytomodulin” and has the sequence A-N-V-A-E-N-A (SEQ ID NO: 1). Cytomodulin when added to cells in culture in the concentration range 10⁻⁹ to 10⁻⁶ M (1.4 pg/mL to 1400 pg/mL), elicits certain highly specific TGF-β effects in several different cell types. For example, among the effects observed is the inhibition of DNA synthesis in Mv-1-Lu mink lung epithelial cells, the growth and colony formation by NRK-49 F fibroblasts in soft agar, and the induction of increased expression of type I collagen in primary cultures of neo-natal human dermal fibroblasts. Moreover, results with human osteogenic sarcoma (HOS) cell line indicate that cytomodulin also may be a mimic for other members of the TGF-β superfamily, such as bone morphogenic proteins (BMPs) and osteogenic protein (OPs), as evidenced by its ability to specifically stimulate markers (alkaline phosphatase and osteonectin) characteristic of the osteoblast phenotype.

Other exemplary TGF-β mimics useful in the methods of the invention are described in U.S. Pat. No. 6,638,912 and are also described in the Examples.

Peptide TGF-β mimics useful in the invention include peptides having at least 70%, at least 80%, at least 90%, at least 95% or greater than 95% sequence identity to the amino acid sequences disclosed herein. In some embodiments, the invention provides methods and compositions that utilize a peptide having at least 70%, at least 80%, at least 90%, at least 95% or greater than 95% sequence identity to an amino acid sequence comprising one of the amino acid sequences of SEQ ID NOS: 1-42. In some embodiments, the invention provides methods and compositions that utilize a peptide having at least 70%, at least 80%, at least 90%, at least 95% or greater than 95% sequence identity to an amino acid sequence comprising one of the amino acid sequences of SEQ ID NOS: 1, 2, 3, 11, 13, or 14. In some embodiments, the invention provides methods and compositions that utilize a peptide having at least 70%, at least 80%, at least 90%, at least 95% or greater than 95% sequence identity to the amino acid sequence of SEQ ID NO: 1.

Percent sequence identity is determined by conventional methods. See, for example, Altschul et al., Bull. Math. Bio. 48:603 (1986), and Henikoff and Henikoff, Proc. Natl. Acad. Sci. USA 89:10915 (1992). Briefly, two amino acid sequences are aligned to optimize the alignment scores using a gap opening penalty of 10, a gap extension penalty of 1, and the “BLOSUM62” scoring matrix of Henikoff and Henikoff (ibid.). The percent identity is then calculated as: ([Total number of identical matches]/[length of the longer sequence plus the number of gaps introduced into the longer sequence in order to align the two sequences])(100).

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of a putative MMP-1 variant. The FASTA algorithm is described by Pearson and Lipman, Proc. Nat'l Acad. Sci. USA 85:2444 (1988), and by Pearson, Meth. Enzymol. 183:63 (1990). Briefly, FASTA first characterizes sequence similarity by identifying regions shared by the query sequence (e.g., SEQ ID NO:1) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, SIAM J. Appl. Math. 26:787 (1974)), which allows for amino acid insertions and deletions. Illustrative parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

The present invention also includes peptides having a conservative amino acid change, compared with an amino acid sequence disclosed herein. Many such changes have been described specifically. More generally, for example, variants can be obtained that contain one or more amino acid substitutions of SEQ ID NO:1-42, in which an alkyl amino acid is substituted for an alkyl amino acid in a TGF-β mimic peptide amino acid sequence, an aromatic amino acid is substituted for an aromatic amino acid in a TGF-β mimic peptide amino acid sequence, a sulfur-containing amino acid is substituted for a sulfur-containing amino acid in a TGF-β mimic peptide amino acid sequence, a hydroxy-containing amino acid is substituted for a hydroxy-containing amino acid in a TGF-β mimic peptide amino acid sequence, an acidic amino acid is substituted for an acidic amino acid in a TGF-β mimic peptide amino acid sequence, a basic amino acid is substituted for a basic amino acid in TGF-β mimic peptide amino acid sequence, or a dibasic monocarboxylic amino acid is substituted for a dibasic monocarboxylic amino acid in a TGF-β mimic peptide amino acid sequence. Among the common amino acids, for example, a “conservative amino acid substitution” is illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. The BLOSUM62 table is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915 (1992)). Accordingly, the BLOSUM62 substitution frequencies can be used to define conservative amino acid substitutions that may be introduced into the amino acid sequences of the present invention. Although it is possible to design amino acid substitutions based solely upon chemical properties (as discussed above), the language “conservative amino acid substitution” preferably refers to a substitution represented by a BLOSUM62 value of greater than -1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

It also will be understood that amino acid sequences may include additional residues, such as additional N- or C-terminal amino acids, and yet still be essentially as set forth in one of the sequences disclosed herein, so long as the sequence retains sufficient biological protein activity to be functional in the compositions and methods of the invention.

A specialized kind of insertional variant is the fusion protein. It is contemplated that the entire TGF-β mimic peptide or a fragment of the TGF-β mimic peptide may be used to construct a fusion protein to enhance tissue specific or cell specific functions of the TGF-β mimic peptide useful in the invention.

A fusion protein generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes such as a hydrolase, glycosylation domains, cellular targeting signals or transmembrane regions.

2. Synthesis

For TGF-β mimics which consist of or include peptides, the peptide may be synthesized by any suitable method for producing peptides of a given sequence. Preferably, peptides of the present invention can be synthesized by various suitable methods that are well known in the art, preferably by solid phase synthesis, manual or automated, as first developed by Merrifield and described by Stewart et al. in Solid Phase Peptide Synthesis (1984). Chemical synthesis joins the amino acids in the predetermined sequence starting at the C-terminus. Basic solid phase methods require coupling the C-terminal protected α-amino acid to a suitable insoluble resin support. Amino acids for synthesis require protection on the α-amino group to ensure proper peptide bond formation with the preceding residue (or resin support). Following completion of the condensation reaction at the carboxyl end, the α-amino protecting group is removed to allow the addition of the next residue. Several classes of α-protecting groups have been described, see Stewart et al. in Solid Phase Peptide Synthesis (1984), with the acid labile, urethane-based tertiary-butyloxycarbonyl (Boc) being the historically preferred. Other protecting groups, and the related chemical strategies, may be used, including the base labile 9-fluorenylmethyloxycarbonyl (FMOC). Also, the reactive amino acid sidechain functional groups require blocking until the synthesis is completed. The complex array of functional blocking groups, along with strategies and limitations to their use, have been reviewed by Bodansky in Peptide Synthesis (1976) and, Stewart et al. in Solid Phase Peptide Synthesis (1984).

Solid phase synthesis is initiated by the coupling of the described C-terminal α-protected amino acid residue. Coupling requires activating agents, such as dicyclohexycarbodiimide (DCC) with or without 1-hydroxybenzo-triazole (HOBT), diisopropylcarbodiimide (DIIPC), or ethyldimethylaminopropylcarbodiimide (EDC). After coupling the C-terminal residue, the α-amino protected group is removed by trifluoroacetic acid (25% or greater) in dichloromethane in the case of acid labile tertiary-butyloxycarbonyl (Boc) groups. A neutralizing step with triethylamine (10%) in dichloro-methane recovers the free amine (versus the salt). After the C-terminal residue is added to the resin, the cycle of deprotection, neutralization and coupling, with intermediate wash steps, is repeated in order to extend the protected peptide chain. Each protected amino acid is introduced in excess (three to five fold) with equimolar amounts of coupling reagent in suitable solvent. Finally, after the completely blocked peptide is assembled on the resin support, reagents are applied to cleave the peptide form the resin and to remove the side chain blocking groups. Anhydrous hydrogen fluoride (HF) cleaves the acid labile tertiary-butyloxycarbonyl (Boc) chemistry groups. Several nucleophilic scavengers, such as dimethylsulfide and anisole, are included to avoid side reactions especially on side chain functional groups.

Slight amino acid modifications to a peptide TGF-β mimic sequence will not affect the peptide's ability to form suitable TGF-β mimics. These modifications include techniques to confer resistance to enzymatic degradation such as adding blocking groups to both the N- and C-terminal residues. Another method for preventing degradation and premature clearance by the renal system is the use of unnatural amino acid substitutes in the peptide sequence. For example, N-methyl-alanine is often substituted for alanine and α-amino isobutryic acid and α-amino butyric acid are substitutes for bulky hydrophobic amino acids.

Recombinant techniques, as known in the art, may also be used to produce peptides suitable for the methods of the invention. Naturally-occurring proteins may be cleaved to produce a desired TGF-β mimic. Methods of designing and screening small molecules may also be used. Methods to generate and screen peptidomimetics may also be useful in producing TGF-β mimics. Substances that are a mixture of peptide and peptidormimetics may also be used.

II. Methods of Cosmetic Use

The methods of the invention have wide applicability to cosmetic conditions. The mode of administration for cosmetic applications is typically topical, but administration and dosage regimens will vary depending on the cosmetic condition whose modulation is sought.

The present invention provides methods, compositions, and kits for cosmetic use with individuals. The term “individual” as used herein includes humans as well as other mammals. In some embodiments, the compositions, methods, and/or kits are used to provide a cosmetic treatment to an invividual desiring and/or in need of cosmetic treatment (e.g., young children subject to burn or other scarring may not desire treatment but may nonetheless be in need of treatment). The term “treating” or “treatment” as used herein includes achieving a cosmetic benefit. By cosmetic benefit is meant any desired modulation of the cosmetic condition being treated. For example, in an individual with wrinkling, cosmetic benefit includes eradication or lessening of the appearance of wrinkling. Also, a cosmetic benefit is achieved with the eradication or amelioration of one or more of the psychological symptoms associated with the underlying condition such that an improvement is observed in the patient, notwithstanding the fact that the patient may still be affected by the cosmetic condition. For example, a TGF-β mimic provides cosmetic benefit not only when a cosmetic defect is eradicated, but also when an improvement is observed in the individual with respect to the cosmetic defect and its attendant consequences, such as psychological consequences. In some cases, methods and compositions of the invention may be directed at achieving a prophylactic benefit. A “prophylactic,” or “preventive” effect includes prevention of a condition, retarding the progress of a condition (e.g., skin aging), or decreasing the likelihood of occurrence of a condition. As used herein, “treating” or “treatment” includes prophylaxis.

As used herein, the term “effective amount” encompasses an amount sufficient to effect beneficial or desired cosmetic results. An effective amount can be administered in one or more administrations. In terms of cosmetic treatment, an “effective amount” of a TGF-β mimic of the invention is an amount that is sufficient to palliate, ameliorate, stabilize, reverse or slow the progression of a cosmetic condition, or to provide a desired effect such as cosmetic augmentation of a soft tissue. An “effective amount” may be of a TGF-β mimic used alone or in conjunction with one or more agents used to modulate a cosmetic condition.

1. Methods of Cosmetic Treatment

The skin is subject to a number of cosmetic conditions that result in alterations of function and/or appearance that are considered undesirable, and whose manifestation can lead to psychological discomfort as well as, in some cases, physiological discomfort or harm. In some cases, although no defect is present, it is nonetheless desirable to the individual to augment or alter the skin in such a way as to produce a cosmetically pleasing effect.

Exemplary cosmetic conditions that may be modulated by the methods of the invention include, but are not limited to, skin aging, cosmetic defect, undesired pigmentation, and post-cosmetic procedure damage.

Skin aging includes chronological aging as well as photoaging, and may appear as wrinkling, lack of elasticity (e.g. sagging), uneven pigmentation, thinning of the skin and/or collagen so that veins and other underlying structures become more prominent, and the like. Skin aging is a major example of a skin condition that involves a decrease in cell proliferation and in cell function. As used herein, “skin aging” refers to alterations in the appearance and function of skin that occur with aging, such as wrinkling, loss of elasticity, sagging, uneven pigmentation (e.g., “age spots” or “liver spots”), and loss of underlying tissue mass. Such conditions may be accelerated and/or exacerbated by exposure to ultraviolet radiation (“photoaging”) and other environmental conditions. With age and/or exposure to UV radiation, the epidermis thins and the skin appendages atrophy. Hair becomes sparse and sebaceous secretions decrease, with consequent susceptibility to dryness, chapping, and fissuring. The dermis diminishes with loss of elastic and collagen fibers. Moreover, keratinocyte proliferation (which is indicative of skin thickness and skin proliferative capacity) decreases with age.

An increase in keratinocyte proliferation and collagen production is believed to counteract skin aging, i.e., wrinkles, thickness, elasticity and repair. According to the present invention, a TGF-β mimic can be used cosmetically to counteract, at least for a time, the effects of aging on skin. A formulation containing a TGF-β mimic may be applied topically in areas where it is desired to counteract skin aging.

Also included in the skin conditions that may be treated by the methods of the invention are cosmetic defects that, while not pathological or physiologically harmful, may nonetheless cause psychological distress, in some cases to the extreme. In these cases it is desirable to correct a particular feature or features causing distress or, alternatively, enhance a feature considered desirable. In addition to skin aging, such conditions include, e.g., striae gravidorum and striae distensiae (“stretch marks), atrophic scarring (e.g., acne scarring), wound (e.g., traumatic wounds, chronic wounds, or burn wounds) or surgical scarring, thickened and cracked skin (especially on the feet), and hair loss.

In the latter embodiments, the invention relates to the use of TGF-β preparations to enhance hair growth. Cells from which the hair is produced grow in the bulb of the follicle. They are extruded in the form of fibers as the cells proliferate in the follicle. Hair “growth” refers to the formation and elongation of the hair fiber by the dividing cells. In some embodiments, the methods of the invention provide a means for altering the dynamics of the hair growth cycle to induce proliferation of hair follicle cells, particularly stem cells of the hair follicle. The subject compositions and method can be used to increase hair follicle size and the rate of hair growth in individuals, such as humans, e.g., by promoting proliferation of hair follicle stem cells. In one embodiment, the method comprises administering to the skin in the area in which hair growth is desired an amount of TGF-β mimic sufficient to increase hair follicle size and/or the rate of hair growth in the animal, e.g., human. Typically, the composition will be administered topically as a cream or lotion, and will be applied on a daily basis until hair growth is observed and for a time thereafter sufficient to maintain the desired amount of hair growth.

Undesired pigmentation includes pigmentation over an area of the body that is different than the pigmentation desired by the individual. Undesired pigmentation can be the result of, e.g., photoaging, reaction to inflammation, or reation to trauma such as surgical or accidental skin breakage, and the like. Undesired pigmentation includes altered or undesired pigmentation over small areas such as freckles, as well as altered or undesired pigmentation over larger areas, such as, for example, uneven pigmentation or larger areas of undesired pigmentation.

Further cosmetic uses of the methods of the invention include tissue augmentation through, generally, topical application, such as for lip enhancement. By “augment” is meant to include giving the appearance of greater fullness, generally through an increase in the tissue of the skin or underlying tissue. Any suitable skin area may be selected for augmentation by the methods of the invention.

In addition, the methods of the invention may be employed to enhance and/or accelerate recovery from standard cosmetic procedures, which are in themselves damaging to skin and/or underlying tissues, and which may take undesirably lengthy periods of time for recovery and/or may produce suboptimal results. Such procedures include chemical peel, dermabrasion, laser resurfacing, ablative resurfacing, nonablative resurfacing, photodynamic therapy, noncoherent light phototherapy, breast lift, face lift, eyelid lift, forehead lift, neck lift, thigh lift, buttock lift, tummy tuck, and scar revision. As will be apparent to those of skill in the art, some of these procedures can require further skin firming (e.g., “lifting” procedures) while others are more extensively damaging to the surface of the skin and require assistance for healing in a timely and optimal fashion (e.g., chemical peel, dermabrasion, ablative and non-ablative skin resurfacing). In some embodiments, the methods of the invention provide a method for achieving firming and lifting of the eyelids; this may be done either in place of or in conjunction with a conventional eyelid lift procedure. The methods of the invention may be used in conjunction with both types of procedures to enhance and/or accelerate healing and recovery.

In any of the methods of the invention, a single TGF-β mimic may be used, or more than one TGF-β mimic may be used. Any suitable TGF-β may be used in accordance with the description herein. In some embodiments, the TGF-β mimic used is that of SEQ ID NO: 1. In some embodiments, the TGF-β mimic used is that of SEQ ID NO: 2. In some embodiments, the TGF-β mimic used is that of SEQ ID NO: 3. In some embodiments, the TGF-β mimic used is that of SEQ ID NO: 11. In some embodiments, the TGF-β mimic used is that of SEQ ID NO: 13. In some embodiments, the TGF-β mimic used is that of SEQ ID NO: 14. In some embodiments, a TGF-β of any of SEQ ID NOS: 4-10, 12, or 15-42 may be used. In some embodiments, a combination of TGF-β mimics is used.

In some embodiments, the TGF-β mimic contains a peptide sequence that is at least about 70%, 80%, 90%, or 95% identical to one of SEQ ID NOS: 1-42, or SEQ ID NOS: 1, 2, 3, 1, 13, or 14, or SEQ ID NO: 1.

The TGF-β mimics may be administered in any cosmetically acceptable carrier, as described in more detail below. In embodiments of methods of the invention, the concentration of TGF-β mimic used may be more than about 0.00001, 0.00005, 0.0001, 0.001, 0.01, 0.1, 1, or 5%. In some embodiments, the concentration of the TGF-β mimic is more than about 0.00005%. The concentration of TGF-β mimic may be less than about 0.0001, 0.001, 0.001, 0.01, 0.1, 1, 5, or 10% (all concentration percentages given herein are w/w unless otherwise indicated). In some embodiments, the TGF-β mimic is used at a concentration of about 0.00001% to about 1%; or about 0.00001% to about 0.1%; or about 0.0001% to about 0.01%; or about 0.0005% to about 0.005%; or about 0.0005% to about 0.002%; or about 0.001%. In some embodiments, lower and higher concentrations, e.g. 0.00001-0.001%, and 0.001-0.01% are contemplated. In some embodiments, even higher concentrations may be warranted, e.g. 0.01%-0.1% or 0.1%-1%, or 0.1%-5%, or 0.1%-10%.

Skin coverage may also be described in terms of total ng of TGF-β mimic/cm² of skin; in these terms, a typical coverage per administration would be more than about 3, 6, 60, 600, 6000, 60,000, or 600,000 ng/cm² of skin; less than about 900,000, 600,000, 60,000, 6000, 600, 60, or 6 ng/cm² of skin; or about 6 ng/cm² to about 600 ng/cm² of skin; or about 60 ng/cm² of skin.

The methods of the invention typically utilize topical administration, which may be by any suitable means that brings the TGF-β mimic and, optionally, other cosmetic or dermatological agents, in contact with the surface of the skin, including application as a gel, lotion, cream, liposomal preparation or the like, with or without occlusion, or application as a plaster, patch, mask, glove, or similar device for extended contact with an affected area of skin. For modulation of cosmetic conditions or to produce a desired cosmetic effect, the frequency and duration of administration of a formulation comprising a TGF-β mimic is dependent on factors including the nature of the formulation (e.g., concentration, presence or lack of other cosmetic or dermatological agents, vehicle type), the severity and extent of the condition, and in some cases the judgment of a skin care professional, e.g., a health care professional such as a dermatologist, or a cosmetologist.

Topical application may be more than about once, twice, three times, four times, five times, or six times per week, or more than once, twice, three times, four times, five times, or six times per day. Frequency of application may be less than about twice, three times, four times, five times, or six times per week, or less than about once, twice, three times, four times, five times, or six times per day. Some embodiments of the invention provide a method for cosmetic treatment of the skin of an individual by topical administration of a an effective amount of a TGF-β mimic. In some embodiments, the formulation is administered an average of about once per day; in some embodiments, the formulation is administered an average of about once or twice per day; in some embodiments, the formulation is administered an average of about once to three times per day; in some embodiments, the formulation is administered an average of more than about three times per day. In one embodiment, the formulation is administered an average of about twice per day, typically in the morning upon rising and in the evening before retiring. Topical administration may be without a covering. Alternatively, topical administration may include the use of a covering over the formulation, which may be occlusive or non-occlusive. For example, administration in the evening before retiring may include covering the administered area with an occlusive or non-occlusive covering, which may remain in place during sleep.

The duration of treatment generally will depend on the response of the skin to cosmetic treatment. Treatment may continue at the discretion of the individual being treated. In some cases, administration or application of a formulation containing a TGF-β mimic may be more frequent at the beginning of treatment and less frequent as treatment continues and the condition is ameliorated or the desired effect is achieved. In some cases, treatment may continue indefinitely in order to maintain a condition in abeyance or in an improved state, to delay onset of a cosmetic condition (e.g., skin aging), or to slow the progression of a cosmetic condition. These modifications of frequency and duration are easily accomplished by the individual being treated.

Some embodiments of cosmetic treatment of skin employ topical administration of a lotion, in some embodiments a mixture of emulsifying lanolin alcohols, waxes, and oils (e.g., EUCERIN™) or a mixture of petrolatum or mineral oil, a quaternary ammonium compound, a fatty alcohol, and a fatty ester emollient (e.g., CUREL™, see U.S. Pat. No. 4,389,418), as described below, comprising a TGF-β mimic. In some embodiments, the TGF-β mimic contains a peptide sequence that is at least about 70%, 80%, 90%, or 95% identical to one of SEQ ID NOS: 1-42, or SEQ ID NOS: 1, 2, 3, 1, 13, or 14, or SEQ ID NO: 1. In some embodiments, the methods utilize cytomodulin (SEQ ID NO: 1). The concentration of the TGF-β mimic (e.g., cytomodulin) can be more than about 0.00005%, 0.0001%; 0.001%; 0.01%; 0.1%; 1%, or 10%; the TGF-1 mimic can be at a concentration of less than about 15%, 10%, 1%, 0.1%, 0.01%; 0.001%; or 0.0001%; in some embodiments, about 0.0001% to about 0.01%, in some embodiments about 0.001%. The lotion containing the TGF-β mimic (e.g., cytomodulin) is applied at a frequency of once to three times per day, in some embodiments once per day, until the desired result, e.g., reduction or elimination of wrinkling, sagging, and the like, is observed, followed by topical application once to three times per week, in some embodiments once per week, thereafter.

If the TGF-β mimic is used in combination with another skin care method or composition, any suitable combination of the TGF-β mimic and the additional method or composition may be used. Thus, for example, if use of a TGF-β mimic is in combination with another cosmetic or dermatological agent, the two may be administered simultaneously, consecutively, in overlapping durations, in similar, the same, or different frequencies, etc. In some cases a composition will be used that contains a TGF-β mimic in combination with one or more other cosmetic or dermatological agents.

Other dermatological or cosmetic agents that may be used in methods of the invention are described in more detail below. Dosages, routes of administration, administration regimes, and the like for these agents are well-known in the art.

III. Compositions

Another aspect of the present invention relates to cosmetic compositions containing a TGF-β mimic. In some embodiments, a combination of TGF-β mimics is used. In some embodiments the TGF-β mimic(s) is in combination with other cosmetic or dermatological ingredients, as described herein. Such compositions are used to modulate cosmetic conditions or to produce desirable cosmetic results.

As the cosmetic compositions are for topical use, they need not be sterile; however, if sterility is desired it is readily accomplished by filtration through sterile filtration (0.22 micron) membranes, or by other art-accepted means.

In cosmetic compositions of the invention, the TGF-β mimic may also be formulated as a cosmetically acceptable salt or cosmetically acceptable ester or amide. As used herein, the term “TGF-β mimic” includes all cosmetically acceptable salts or cosmetically acceptable esters or amides thereof. The term “cosmetically acceptable salt” or “cosmetically acceptable ester or amide” means those salts, esters, or amides that retain the cosmetic effectiveness and properties of the compounds used in the present invention. For example, a cosmetically acceptable salt does not interfere with the cosmetically acceptable effect of a TGF-β mimic in modulating an age-related condition such as wrinkling.

TGF-β mimics form cosmetically acceptable salts with organic and inorganic acids and can be administered in salt form or the TGF-β mimic can be amidated. Examples of suitable acids for the formation of cosmetically acceptable salts are hydrochloric, sulfuric, phosphoric, acetic, benzoic, citric, malonic, salicylic, malic, fumaric, succinic, tartaric, lactic, gluconic, ascorbic, maleic, benzene-sulfonic, methane- and ethanesulfonic, hydroxymethane- and hydroxyethane-sulfonic.

Salts may also be formed with suitable organic cosmetically acceptable base addition salts. These organic bases form a class whose limits are readily understood by those skilled in the art. Merely for purposes of illustration, the class may be said to include mono-, di-, and trialkylamines, such as methylamine, dimethylamine, and triethylamine; mono-, di-, or trihydroxyalkylamines such as mono-, di-, and triethanolamine; amino acids such as arginine, and lysine; guanidine; N-methyl-glucosamine; N-methyl-glucamine; L-glutamine; N-methylpiperazine; morpholine; ethylenediamine; N-benzylphenethylamine; tris(hydroxymethyl)aminomethane; and the like. (See, for example, “Pharmaceutical Salts,” J. Pharm. Sci., 66(1), 1-19 (1977), which are acceptable as cosmetically acceptable salts as well.)

If desired, further cosmetic or dermatological ingredients may be incorporated in the formulations. The nature of the other ingredient(s) will depend on the cosmetic condition to be modulated and/or cosmetic result desired. These are described more fully below.

The TGF-β mimic may be used neat (e.g., with an occlusive dressing so that the TGF-β mimic is dissolved or dispersed in perspiration at the site), but generally is prepared in a vehicle suitable for topical administration. Compositions of the invention include TGF-β mimic in a vehicle suitable for topical administration.

Numerous vehicles for topical application of cosmetic compositions are known in the art. See, e.g., Remington's Pharmaceutical Sciences, Gennaro, A R, ed., 20^{th} edition, 2000: Williams and Wilkins PA, USA. All compositions usually employed for topically administering cosmetic compositions may be used, e.g., creams, lotions, gels, dressings, shampoos, tinctures, pastes, ointments, salves, powders, liquid or semiliquid formulation, patches, liposomal preparations, and the like. Application of said compositions may, if appropriate, be by aerosol e.g. with a propellant such as nitrogen carbon dioxide, a freon, or without a propellant such as a pump spray, drops, lotions, or a semisolid such as a thickened composition which can be applied by a swab. In particular compositions, semisolid compositions such as salves, creams, lotions, pastes, gels, ointments and the like will conveniently be used. The TGF-β mimic may optionally be dissolved in a small amount (e.g., less than 100, less than 10, or less than 1 ug/ml TGF-β mimic; in some embodiments, less than 1 ug/ml TGF-β mimic) of an appropriate solvent, such as ethanol or DMSO, before dispersion in the vehicle; however, this is not required.

Compositions known in the art, preferably hypoallergic and pH controlled are especially preferred for topical administration, and include toilet waters, packs, lotions, skin milks or milky lotions. The preparations contain, besides the TGF-β mimic and, optionally, other active ingredients, components usually employed in such preparations. Examples of such components are oils, fats, waxes, surfactants, humectants, thickening agents, antioxidants, viscosity stabilizers, chelating agents, buffers, preservatives, perfumes, dyestuffs, lower alkanols, and the like.

Examples of oils include fats and oils such as olive oil and hydrogenated oils; waxes such as beeswax and lanolin; hydrocarbons such as liquid paraffin, ceresin, and squalene; fatty acids such as stearic acid and oleic acid; alcohols such as cetyl alcohol, stearyl alcohol, lanolin alcohol, and hexadecanol; and esters such as isopropyl myristate, isopropyl palmitate and butyl stearate. As examples of surfactants there may be cited anionic surfactants such as sodium stearite, sodium cetylsulfate, polyoxyethylene laurylether phosphate, sodium N-acyl glutamate; cationic surfactants such as stearyldimethylbenzylammonium chloride and stearyltrimethylammonium chloride; ampholytic surfactants such as alkylaminoethylglycine hydrochloride solutions and lecithin; and nonionic surfactants such as glycerin monostearate, sorbitan monostearate, sucrose fatty acid esters, propylene glycol monostearate, polyoxyethylene oleylether, polyethylene glycol monostearate, polyoxyethylene sorbitan monopalmitate, polyoxyethylene coconut fatty acid monoethanolamide, polyoxypropylene glycol (e.g. the materials sold under the trademark “Pluronic”), polyoxyethylene castor oil, and polyoxyethylene lanolin. Examples of humectants include glycerin, 1,3-butylene glycol, and propylene glycol; examples of lower alcohols include ethanol and isopropanol; examples of thickening agents include xanthan gum, hydroxypropyl cellulose, hydroxypropyl methyl cellulose, polyethylene glycol and sodium carboxymethyl cellulose; examples of antioxidants include butylated hydroxytoluene, butylated hydroxyanisole, propyl gallate, citric acid and ethoxyquin; examples of chelating agents include disodium edetate and ethanehydroxy diphosphate; examples of buffers include citric acid, sodium citrate, boric acid, borax, and disodium hydrogen phosphate; and examples of preservatives are methyl parahydroxybenzoate, ethyl parahydroxybenzoate, dehydroacetic acid, salicylic acid and benzoic acid. These substances are merely exemplary, and those of skill in the art will recognize that other substances may be substituted with no loss of functionality.

For preparing compositions for topical administration, the concentration of TGF-β mimic may be more than about 0.00001, 0.00005, 0.0001, 0.001, 0.01, 0.1, 1, or 5%. In some embodiments, the concentration of the TGF-β mimic is more than about 0.00005%. The concentration of TGF-β mimic may be less than about 0.0001, 0.001, 0.001, 0.01, 0.1, 1, 5, or 10% (all concentration percentages given herein are w/w unless otherwise indicated). In some embodiments, the TGF-β mimic is incorporated at a concentration of about 0.00001% to about 1%; or about 0.00001% to about 0.1%; or about 0.0001% to about 0.01%; or about 0.0005% to about 0.005%; or about 0.0005% to about 0.002%; or about 0.001%. In some embodiments, lower and higher concentrations, e.g. 0.00001-0.001%, and 0.001-0.01% are contemplated. In some embodiments, even higher concentrations may be warranted, e.g. 0.01%-0.1% or 0.1%-1%, or 0.1%-5%, or 0.1%-10%. In some embodiments employing ointments, lotions, or creams, the carrier for example, can contain 1 to 20%, in particular 5 to 15% of a humectant, 0.1 to 10% in particular from 0.5 to 5% of a thickener and water; or said carrier may consist of 70 to 99%, in particular 20 to 95% of a surfactant, and 0 to 20%, in particular 2.5 to 15% of a fat; or 80 to 99.9% in particular 90 to 99% of a thickener; or 5 to 15% of a surfactant, 2-15% of a humectant, 0 to 80% of an oil, very small (<2%) amounts of preservative, coloring agent and/or perfume, and water. In a toilet water, the carrier for example consists of 2 to 10% of a lower alcohol, 0.1 to 10% or in particular 0.5 to 1% of a surfactant, 1 to 20%, in particular 3 to 7% of a humectant, 0 to 5% of a buffer, water and small amounts (<2%) of preservative, dyestuff and/or perfume. In a skin milk, the carrier typically consists of 10-50% of oil, 1 to 10% of surfactant, 50-80% of water and 0 to 3% of preservative and/or perfume.

In some embodiments, compositions of the invention contain a TGF-β mimic that contains a peptide sequence that is at least about 70%, 80%, 90%, or 95% identical to one of SEQ ID NOS: 1-42, or SEQ ID NOS: 1, 2, 3, 1, 13, or 14, or SEQ ID NO: 1. In some embodiments, the compositions contain cytomodulin (SEQ ID NO: 1).

In some embodiments, the lotion in which one or more TGF-β mimics, optionally with other active ingredients, is (dissolved, mixed, or suspended) in a mixture of emulsifying lanolin alcohols, waxes, and oils (e.g., EUCERIN™ Lotion) or a mixture of petrolatum or mineral oil, a quaternary ammonium compound, a fatty alcohol, and a fatty ester emollient (e.g., CUREL™ Lotion), or lotions that are substantially similar in composition.

EUCERIN™ Dry Skin Therapy Original Moisturizing Lotion comprises water, mineral oil, isopropyl myristate, PEG-40 sorbitan peroleate, glyceryl lanolate, sorbitol, propylene glycol, cetyl palmitate, magnesium sulfate, aluminum stearate, lanolin alcohol, BHT, methylchloroisothiazolinone, and methylisothiazolinone. CUREL™ Fragrance Free Daily Moisturizing Lotion comprises water, glycerin, distearyldimonium chloride, petrolatum, isopropyl palmitate, cetyl alcohol, dimethicone, sodium chloride, methylparaben, and propylparaben.

Some embodiments of compositions of the invention comprise a TGF-β mimic in a lotion comprising a mixture of emulsifying lanolin alcohols, waxes, and oils (e.g., EUCERIN™ Dry Skin Therapy Original Moisturizing Lotion) at a concentration greater than about 0.00005%. Some embodiments of compositions of the invention comprise a TGF-β mimic in a lotion comprising a mixture of emulsifying lanolin alcohols, waxes, and oils (e.g., EUCERIN™ Dry Skin Therapy Original Moisturizing Lotion) at a concentration of about 0.0001% to about 0.01%. Some embodiments comprise cytomodulin (SEQ ID NO:1) in a lotion comprising a mixture of emulsifying lanolin alcohols, waxes, and oils (e.g., EUCERIN™ Dry Skin Therapy Original Moisturizing Lotion) at a concentration of about 0.001%.

Some embodiments of compositions of the invention comprise a TGF-β mimic in a lotion comprising a mixture of petrolatum or mineral oil, a quaternary ammonium compound, a fatty alcohol, and a fatty ester emollient (e.g., CUREL™ Fragrance Free Daily Moisturizing Lotion) at a concentration greater than about 0.00005%. Some embodiments of compositions of the invention comprise a TGF-β mimic in a lotion comprising a mixture of petrolatum or mineral oil, a quaternary ammonium compound, a fatty alcohol, and a fatty ester emollient (e.g., CUREL™ Fragrance Free Daily Moisturizing Lotion) at a concentration of about 0.0001% to about 0.01%. Some embodiments comprise cytomodulin (SEQ ID NO:1) in a lotion comprising a mixture of petrolatum or mineral oil, a quaternary ammonium compound, a fatty alcohol, and a fatty ester emollient (e.g., CUREL™ Fragrance Free Daily Moisturizing Lotion) at a concentration of about 0.001%.

In addition, a TGF-β mimic and, optionally, other active ingredients, may be formulated in liposome-containing compositions. Liposomes are artificial vesicles formed by amphipathic molecules such as polar lipids, for example, phosphatidyl cholines, ethanolamines and serines, sphingomyelins, cardiolipins, plasmalogens, phosphatidic acids and cerebrosides. Liposomes are formed when suitable amphipathic molecules are allowed to swell in water or aqueous solutions to form liquid crystals usually of multilayer structure comprised of many bilayers separated from each other by aqueous material (also referred to as coarse liposomes). Another type of liposome known to be consisting of a single bilayer encapsulating aqueous material is referred to as a unilamellar vesicle. If water-soluble materials are included in the aqueous phase during the swelling of the lipids they become entrapped in the aqueous layer between the lipid bilayers.

The incorporation of TGF-β mimics into liposomal preparations suitable for topical application can be achieved by a number of methods. With respect to liposomal preparations, any known methods for preparing liposomes for treatment of a condition may be used. See, for example, Bangham et al., J. Mol. Biol, 23: 238-252 (1965) and Szoka et al., Proc. Natl Acad. Sci. 75: 4194-4198 (1978). Ligands may also be attached to the liposomes to direct these compositions to particular sites of action.

Liposomes containing a TGF-β mimic and, optionally, other ingredients can be employed directly or they can be employed in a suitable pharmaceutically acceptable carrier for topical administration. The viscosity of the liposomes can be increased by the addition of one or more suitable thickening agents such as, for example xanthan gum, hydroxypropyl cellulose, hydroxypropyl methyl cellulose and mixtures thereof. The aqueous component may consist of water alone or it may contain electrolytes, buffered systems and other ingredients, such as, for example, preservatives. Suitable electrolytes which can be employed include metal salts such as alkali metal and alkaline earth metal salts. Exemplary metal salts are calcium chloride, sodium chloride and potassium chloride. The concentration of the electrolyte may vary from zero to 260 mM, preferably from 5 mM to 160 mM. The aqueous component is placed in a suitable vessel which can be adapted to effect homogenization by effecting great turbulence during the injection of the organic component. Homogenization of the two components can be accomplished within the vessel, or, alternatively, the aqueous and organic components may be injected separately into a mixing means which is located outside the vessel. In the latter case, the liposomes are formed in the mixing means and then transferred to another vessel for collection purpose.

The organic component consists of a suitable non-toxic, cosmetically acceptable solvent such as, for example ethanol, glycerol, propylene glycol and polyethylene glycol, and a suitable phospholipid which is soluble in the solvent. Suitable phospholipids which can be employed include lecithin, phosphatidylcholine, phosphatidylserine, phosphatidylethanolamine, phosphatidylinositol, lysophosphatidylcholine and phosphatidyl glycerol, for example. Other lipophilic additives may be employed in order to selectively modify the characteristics of the liposomes. Examples of such other additives include stearylamine, phosphatidic acid, tocopherol, cholesterol and lanolin extracts. In addition, other ingredients which can prevent oxidation of the phospholipids may be added to the organic component. Examples of such other ingredients include tocopherol, butylated hydroxyanisole, butylated hydroxytoluene, ascorbyl palmitate and ascorbyl oleate. Preservatives such a benzoic acid, methyl paraben and propyl paraben may also be added.

Although TGF-β mimic are generally capable of penetrating cell membranes and reaching the deep layers of skin, it may be useful in some embodiments to also include a penetration enhancer in the formulations of the invention. A penetration enhancer is a substance that improves cutaneous penetration of a bioactive substance. Suitable penetration enhancers include, for example, dimethyl sulfoxide (DMSO), DMSO-like compounds, ethanolic compounds, pyroglutamic acid esters and other solvents or compounds known to those skilled in the pharmaceutical art which facilitate dermal penetration of the drugs or chemicals chosen for the pharmaceutical composition. Other penetration enhancers include amphiphiles such as L-amino acids, anionic surfactants, cationic surfactants, amphoteric surfactants, nonionic surfactants, fatty acids and alcohols. Additional penetration enhancers are disclosed in Remington: The Science and Practice of Pharmacy, 19th Edition (1995) on page 1583. The penetration enhancer chosen and the relative proportion of the penetration enhancer with respect to the active drugs or chemicals depends on the desired rate of delivery of the drugs or chemicals into the skin, which in turn depends on the condition being treated and the outcome sought. More specifically, the type and amount of enhancer is chosen so that a sufficiently high concentration of active drugs or chemicals is attained in the skin to treat the condition within the time period considered desirable.

Apart from the above-described compositions, use may be made of covers, e.g. plasters, bandages, dressings, gauze pads, patches and the like, containing an appropriate amount of a TGF-β mimic and, optionally, other ingredients. In some cases use may be made of plasters, bandages, dressings, gauze pads, patches and the like which have been impregnated with a topical formulation containing the therapeutic formulation.

Additional cosmetic and dermatological agents. Other cosmetic or dermatological agents may be included in methods and formulations of the invention. A “cosmetic or dermatological agent” as used herein, includes any substance whose administration for treatment of a cosmetic condition or to achieve a desired cosmetic effect, results in an status of the condition that is better than the status that would exist without the use of the cosmetic or dermatological agent.

Anti-wrinkling agents are one form of cosmetic or dermatological agents. Anti-skin wrinkling agents include a variety of agents, often in combination, that prevent or treat wrinkling through a variety of actions. Many approaches are taken to reduce the appearance of facial and other wrinkles based on the understanding of the molecular basis of wrinkle formation. Such treatments include cosmetic products, drug therapy and surgical procedures. For example, many cosmetic products contain hydroxy acids, which may stimulate collagen synthesis. Another common treatment utilizes retinol, retinoic acid, retinol palmitate, a derivative of vitamin A, (or prescribed versions, Retin-A and Renova) which may directly or indirectly stimulate collagen production or retard collagen degradation. Bicyclic aromatic compounds with retinoid-type activity, which are useful in particular in preventing or treating various keratinization disorders, are described in EP 679 630. These compounds are particularly active for repairing or combating chronological or actinic ageing of the skin, for example such as in anti-wrinkle products. Antioxidants such as vitamin C and E and coenzyme Q-10 are believed to counteract free radicals, which damage cells and cause aging and have been used in treatments of wrinkles. Recently, the FDA approved cosmetic use of Botox (an extremely purified form of botulinum toxin) to treat glabella frown lines.

Thus cosmetic or dermatological agents that complement cosmetic treatment of skin with the methods or compositions of the invention include, alone or in combination, the bicyclic aromatic compounds defined above, other compounds which have retinoid-type activity, free-radical scavengers, hydroxy or keto acids or derivatives thereof.

The term “free-radical scavenger” refers to, for example, α-tocopherol, superoxide dismutase, ubiquinol (e.g., coenzyme Q10) or certain metal-chelating agents. Hydroxy acids include, e.g., alpha-hydroxy acids such as lactic acid and glycolic acid or beta-hydroxy acids such as salicylic acid and salicylic acid derivatives such as the octanoyl derivative; other hydroxy acids and keto acids include malic, citric, mandelic, tartaric or glyceric acids or the salts, amides or esters thereof.

Other anti-wrinkling agents and anti-skin aging agents useful in the invention include sulfur-containing D and L amino acids and their derivatives and salts, particularly the N-acetyl derivatives, a preferred example of which is N-acetyl-L-cysteine; thiols, e.g. ethane thiol; fat-soluble vitamins, ascorbyl palmitate, ceramides, pseudoceramides (e.g., pseudoceramides described in U.S. Pat. Nos. 5,198,210; 4,778,823; 4,985,547; 5,175,321, all of which are incorporated by reference herein), phospholipids (e.g., distearoyl lecithin phospholipid), fatty acids, fatty alcohols, cholesterol, plant sterols, phytic acid, lipoic acid; lysophosphatidic acid, and skin peel agents (e.g., phenol and the like), and mixtures thereof. Preferred fatty acids or alcohols are those that have straight or branched alkyl chains containing 12-20 carbon atoms. A particularly preferred fatty acid is linoleic acid since linoleic acid assists in the absorption of ultraviolet light and furthermore is a vital component of the natural skin lipids. Other non-limiting examples of suitable anti-wrinkle actives for use herein are described in U.S. Pat. No. 6,217,888, which description is incorporated herein by reference.

Compositions for cosmetic treatment of skin may further include sunscreens to lower skin's exposure to harmful UV rays. Sunscreens include those materials commonly employed to absorb or block ultraviolet light. Illustrative compounds are the derivatives of PABA, cinnamate and derivatives of salicylate (other than ferulyl salicylate). For example, octyl methoxycinnamate and 2-hydroxy-4-methoxy benzophenone (also known as oxybenzone) can be used. Octyl methoxycinnamate and 2-hydroxy-4-methoxy benzophenone are commercially available under the trademarks, PARSOL MCX and BENZOPHENONE-3, respectively. Dermascreen may also be used.

Many other sunscreens are known to those of skill in the art. In some embodiments, sunscreens are FDA-approved or approved for use in the European Union. For example, FDA-approved sunscreens may be used, singly or, preferably, in combination. See, e.g., U.S. Pat. Nos. 5,169,624; 5,543,136; 5,849,273; 5,904,917; 6,224,852; 6,217,852; and Segarin et al., chapter Vil, pages 189 of Cosmetics Science and Technology, and Final Over-the-Counter Drug Products Monograph on Sunscreens (Federal Register, 1999:64:27666-27963), all of which are incorporated herein by reference. The exact amount of sunscreen employed in-the compositions can vary depending upon the degree of protection desired from the sun's UV radiation.

Cosmetic compositions of the invention may further include anti-acne agents. Anti-acne agents include benzoyl peroxide, antibiotics, e.g., erythromycin, clindamycin phosphate, 5,7-dichloro-8-hydroxyquinoline, resorcinol, resorcinol acetate, salicylic acid, azaleic acid, long chain dicarboxylic acids, sulfur, zinc, retinoids, antiandrogens, and various natural agents such as those derived from green tea, tea tree oil, and mixtures thereof. Other non-limiting examples of suitable anti-acne agents for use herein are described in U.S. Pat. No. 5,607,980, which description is incorporated herein by reference.

Other cosmetic and dermatological agents include anticellulite agents. Anticellulite agents include isobutylmethylxanthine, caffeine, theophylline, theobromine, aminophylline, yohimbine, and mixtures thereof.

Yet other cosmetic or dermatological agents that complement cosmetic treatment of skin include α-interferon, estradiol; progesterone; pregnanalone; methylsolanomethane (MSM); copper peptide (copper extract); plankton extract (phytosome); broparoestrol; estrone; adrostenedione; androstanediols; etc.

The compositions of the present invention may contain a wide range of additional components. The CTFA Cosmetic Ingredient Handbook, Seventh Edition, 1997 and the Eighth Edition, 2000, which are incorporated by reference herein in their entirety, describes a wide variety of ingredients commonly used in skin care compositions, which are suitable for use in the compositions of the present invention. Other topically-applied compounds are listed in Remington's Pharmaceutical Sciences, 20th Ed., Lippincott Williams & Witkins, Baltimore, Md. (2000) (hereinafter Remington's), U.S. Pharmacopeia and National Formulary, The United States Pharmacopeial Convention, Inc., Rockville, Md. and Physician's Desk Reference, Medical Economics Co., Inc., Oradell, N.J. incorporated herein by reference. The concentration of the other active ingredient in formulations provided by the invention is that which provides an effective amount of the other active ingredient; these concentrations are well-known in the art. See, e.g., the above references, as well as Textbook of Dermatology, Champion, Burton, Burns, and Bretnach, eds., Blackwell Publishing, 1998.

IV. Kits of the Invention

In still another aspect, the present invention provides kits for the cosmetic treatment of skin or to produce a desired cosmetic result. These kits comprise a TGF-β mimic or TGF-β mimic composition or compositions described herein, in a container or containers which are held in suitable packaging. In some embodiments the kits further contain instructions teaching the use of the kit according to the various methods and approaches described herein. Such kits may also include information, such as scientific literature references, package insert materials, cosmetic trial results, and/or summaries of these and the like, which indicate or establish the activities and/or advantages of the composition. Such information may be based on the results of various studies, for example, studies using experimental animals involving in vivo models and studies based on human cosmetic or clinical trials. Kits described herein can be provided, marketed and/or promoted to health care providers (e.g., dermatologists and other physicians), skin care appearance care providers, including cosmetologists, hair stylists, and the like. Kits for cosmetic use may also be provided, marketed and/or promoted directly to consumers. Kits may be marketed in spas and retail outlets.

### EXAMPLES

### Example 1

### Inhibition of DNA Synthesis of Mv-1-Lu Mink Lung Epithelial Cells

The effect of TGF-β and cytomodulin were evaluated by determining the rate of [¹H]thymidine incorporation into total acid-insoluble DNA and cell number. See generally, Sampath et al., J. Biol. Chem., 267, pp. 20352-20362 (1992). DNA synthesis rates were determined in triplicate cultures after 24 hour treatment with various concentrations (10⁻⁹ M to 10⁻⁶ M) of either TGF-β or cytomodulin (which was synthesized by the Merrifield method) by adding [methyl-³H]thymidine (2uCi/ml, 80 Ci/mmol) for 6 hours before the termination of the culture. Incorporation was terminated by aspiration of the medium, and after washing three times with phosphate-buffered saline, the trichloroacetic acid (10%)-precipitated radioactive DNA was extracted with 1.0% (w/v) sodium dodecyl sulfate, 0.1 M NaOH and quantitated by liquid scintillation counting. For cell number determination, 1×10⁵ cells were plated in flasks in MEM containing 10% FBS, and after 24 hours, the growth medium was replaced with serum-free medium containing various conceptions of TGF-β and cytomodulin. Triplicate cultures were harvested every 24 hours for the duration of 7 days, and the cell number was determined by counting cells released by trypsin digestion in a fixed volume hemacytometer.

The growth inhibition curve for cytomodulin were similar to that observed for TGF-β at the same concentration range.

### Example 2

### Growth and Colony Formation by NRK-49 F Fibroblasts in Soft Agar

The original assay for TGF-β, the ability to promote anchorage independent growth of normal fibroblasts is still one of the hallmarks of TGF-β activity. NRK-49 F fibroblasts were grown at 37° C. in DEM supplemented with 10% fetal calf serum. The experiments were performed with culture medium, 10 ng/mg epidermal growth factor (EGF), and 10 ng/ml platelet-derived growth factor (PDGF); however, unlike TGF-β, which does not induce colony formation in the absence of these factors (see, for example, Massagu, J. Biol. Chem., 259, pp. 9756-9761 (1984)), cytomodulin did induce colony formation without these two growth factors. To this, either 100 nM TGF-β (positive control) or 100 nM cytomodulin was added. NRK49 F fibroblasts (5×10⁴ cells/mi) were mixed with 0.3% agar were plated on the bottom of 35 mm culture dishes. Colony formation was observed starting on day 3 of culture.

As expected no colonies were formed in those cultures containing only the basic medium. Also, as expected, colonies with TGF-β grew colonies. Surprisingly, the cytomodulin cultures also formed colonies to approximately the same extent as the TGF-β cultures. The growth characteristics of the colonies over time were similar between TGF-β and cytomodulin cultures.

### Example 3

### Further TGF-β Mimics

FIGS. 1A and 1B show the atomic coordinates of the bioactive structure of cytomodulin (atoms 1-101). Thus, the structure represented by FIGS. 1A and 1B, describes one of the possible structures consistent with TGF-β activity

Using the three dimensional structure of cytomodulin (FIGS. 1A and 1B) as a guide, cytomodulin analogs were designed and tested for TGF-β activity. An exemplary general formula to produce a peptide with atomic coordinates substantially the same as that of FIGS. 1A and 1B, which was used in the construction of these two cytomodulin analogs, was AAᵢ-AAᵢ₊₁-AAᵢ₊₂ . . . -AAᵢ₊ₙ:
(1) a hydrophobic or neutral amino acid at position i;
(2) a branched hydrophobic at position i+1;
(3) a small aliphatic at position i+2, where positions i+1 and i+2 together form the U-bend (β-bend) structure; and
(4) a side-chain possessing a hydrogen bond acceptor shortly thereafter (at position AAᵢ₊ₙ)

Initially, two cytomodulin analogs, L-I-A-E-A-K (SEQ ID NO: 2 or L2) and L-I-A-P-E-A (SEQ ID NO:3 or L1) were synthesized and tested. In both peptides, -V-A- was replaced by -I-A- and the first two N-terminal amino acid sequence of cytomodulin was replaced with leucine. In SEQ ID NO:2, glutamic acid is at position i+3 since it is the first side-chain after the U (β)-bend structure. In SEQ ID NO:3, proline is at position i+3. Since proline does not have a “side-chain,” the glutamic acid was placed at position i+4.

Both cytomodulin analogs, L1 and L2, displayed at least as much TGF-β like activity as cytomodulin. They promoted the growth of NRK-49F cells in soft agar and inhibited the proliferation of MV-1-Lu cells. They increased the expression of type I collagen and TGF-β and decreased the expression of collagenase in human dermal fibroblasts. Moreover, as with cytomodulin, L1 and L2 also increased the expression of type I collagen, TGF-β and alkaline phosphatase in HOS cells.

Further analogs of SEQ ID NO:2 and SEQ ID NO:3 were then made:

| | |
|---|---|
| L-(Aib)-A-E-A-K | (SEQ ID NO:4) |
| L-I-(Nme-A)-E-A-K | (SEQ ID NO:5) |
| L-(Abu)-A-E-A-K | (SEQ ID NO:6) |
| G-G-Q-I-A-N-I | (SEQ ID NO:7) |
| E-G-I-A-G-K | (SEQ ID NO:8) |
| L-I-A-D-A-K | (SEQ ID NO:9) |
| L-I-A-N-A-K | (SEQ ID NO:10) |
| L-I-A-E-A-A | (SEQ ID NO:11) |
| L-I-A-Q-A-K | (SEQ ID NO:12) |
| L-I-A-G-G-E | (SEQ ID NO:13) |
| L-I-A-G-E-G | (SEQ ID NO:14) |
| A-N-V-A-E-K | (SEQ ID NO:15) |
| L-I-A-K-G-K | (SEQ ID NO:16) |

Of the non-standard amino acids, Aib is α-amino isobutyric acid, Nme-Ala is N-methyl alanine and Abu is α-amino butyric acid.

SEQ ID NOs:4-6, which are minor variants of SEQ ID NO:2, mimicked the biological activities of TGF-β and cytomodulin as shown by the inhibition of the proliferation of Mv-1-Lu epithelial cells and increased expression of collagen I and TGF-β in HOS cells. Sample thymidine incorporation data for SEQ ID NOs:4-6 are shown in Table 1.

TABLE 1
Inhibition of Incorporation of ³H-thymidine inMV-1 Lu cells
in the Presence of Test Peptides

| Control | ³H-Radioactivity, 10.sup.3 dpm |
|---|---|
| (No peptides added) | (% Inhibition) 3.57 (−) |
| (IV) LAibAEAK (SEQ ID NO: 4) | |
| 1 mM | 2.60 (27%) |
| 5 nM | 2.31 (35%) |
| 50 nM | 1.94 (46%) |
| 100 nM | 1.44 (60%) |
| 500 nM | 1.61 (55%) |
| (V) LINmeAEAK (SEQ ID NO: 5) | |
| 1 nM | 1.61 (47%) |
| 5 nM | 1.76 (42%) |
| 50 nM | 1.60 (41%) |
| 100 nM | 1.81 (40%) |
| 500 nM | 1.42 (53%) |
| (VI) LAbuAEAK (SEQ ID NO: 6) | |
| 1 nM | 2.02 (33%) |
| 5 nM | 1.97 (35%) |

However, SEQ ID NOs:7-8 did not display significant TGF-β activity and thus, as that term is defined herein, and by the assays used, would not be defined as TGF-β mimics since, although they possessed an atomic structure substantially the same as that shown in FIGS. 1A and 1B, they did not show TGF-β activity. This was not unexpected given the working model; e.g., although a glutamic acid is present in SEQ ID NO:8, it is on the N-terminal side of the β-bend and not on the C-terminal side as with cytomodulin, L1, and L2.

Inhibition of ³H-thymidine incorporation results for SEQ ID NOs:9-16 are shown in Table 2. The numbers shown at the various concentration are the ratio of the inhibition rate of the peptide being tested over the inhibition rate of cytomodulin (SEQ ID NO: 1) at the same concentration. Because cytomodulin inhibits the proliferation of MV-1-Lu cells at least as much as TGF-β, cytomodulin and not TGF-β was used as a control.

TABLE 2

| | | (Inhib by new peptide/ | | | |
|---|---|---|---|---|---|
| | | Inhib by cytomodulin) | | | |
| Peptide | | Concentration (nM) | | | |
| Composition | SEQ ID NO | 1 | 10 | 100 | 1000 |
| LIADAK | SEQ ID NO: 9 | 0.45 | 0.87 | 1.14 | 1.25 |
| LIANAK | SEQ ID NO: 10 | 1.70 | 2.00 | 1.43 | 3.05 |
| LIAEAA | SEQ ID NO: 11 | 1.16 | 1.00 | 1.17 | 1.85 |
| LIAQAK | SEQ ID NO: 12 | 0.90 | 1.10 | 0.80 | 1.56 |
| LIAGGE | SEQ ID NO: 13 | 1.10 | 1.33 | 1.45 | 1.90 |
| LIAGEG | SEQ ID NO: 14 | 0.66 | 0.85 | 1.41 | 1.88 |
| ANVAEK | SEQ ID NO: 15 | — | 0.80 | 1.00 | — |
| LIAKGK | SEQ ID NO: 16 | — | 0.65 | 0.67 | — |

As illustrated by Table 2, all peptides with sequences represented by SEQ ID NOs:9-16 inhibited at least some amount of thymidine uptake. Thus, all peptides with sequences represented by SEQ ID NOs:9-16 are considered TGF-β mimics.

Further analogs were constructed (SEQ ID NOS: 17-42):

| | |
|---|---|
| SEQ ID NO 17: | Gly Thr Pro Gly Pro Gln Gly Ile Ala |
| | Gly Gln Arg Gly Val Val |
| SEQ ID NO 18: | Ile Xaa Ala Glu Ala Lys |
| SEQ ID NO 19: | Leu Xaa Ala Glu Ala Lys |
| SEQ ID NO 20: | Leu Pro Ala Glu Ala Lys |
| SEQ ID NO 21: | Leu Ile Pro Glu Ala Lys |
| SEQ ID NO 22: | Leu Ile Xaa Glu Ala Lys |
| SEQ ID NO 23: | Leu Ile Ala Xaa Glu Ala |
| SEQ ID NO 24: | Ile Trp Gly Leu Asp Gly Xaa Lys |
| SEQ ID NO 25: | Trp Ile Ala Leu Glu Gly Xaa Lys |
| SEQ ID NO 26: | Gly Pro Gln Gly Ile Ala Gly Gln Arg |
| SEQ ID NO 27: | Gln Gly Ile Ala Gly Gln |
| SEQ ID NO 28: | Gln Gly Ile Ala Gly Gln Arg |
| SEQ ID NO 29: | Phe Gly Ile Ala Gly Phe |
| SEQ ID NO 30: | Gly Ile Ala Gly Gln |
| SEQ ID NO 31: | Gln Gly Ala Ile Ala Gln |
| SEQ ID NO 32: | Phe Gly Ile Ala Gly Phe |
| SEQ ID NO 33: | Cys Gly Ile Ala Gly Cys |
| SEQ ID NO 34: | Glu Gly Ile Ala Gly Lys |
| SEQ ID NO 35: | Xaa Ile Ala Ala |
| SEQ ID NO 36: | Ile Ala Xaa |
| SEQ ID NO 37: | Xaa Ile Ala Xaa |
| SEQ ID NO 38: | Ile Ile Xaa Glu Ala Lys |
| SEQ ID NO 39: | Leu Ile Xaa Glu Ala Lys |
| SEQ ID NO 40: | Leu Ile Ala Xaa Ala Lys |
| SEQ ID NO 41: | Leu Ile Ala Pro Xaa Ala |
| SEQ ID NO 42: | Leu Ile Ala Xaa Ala Lys |

Tests for bioactivity were conducted as described above for DNA inhibition. In summary, the cytomodulin analogs listed in Table 3 were all found active as agonists and thus, as defined herein, are considered TGF-β mimics.

TABLE 3

| | | |
|---|---|---|
| | | SEQ ID |
| SEQUENCE | SEQUENCE SYMBOL | NO |

| | | |
|---|---|---|
| Ala-Asn-Val-Ala-Glu- | A-N-V-A-E-N-A | 1 |
| Asn-Ala | | |
| Leu-Ile-Ala-Pro-Glu- | L-I-A-P-E-A | 3 |
| Ala | | |
| Leu-Ile-Ala-Glu-Ala- | L-I-A-E-A-K | 2 |
| Lys | | |
| Ile-Aib-Ala-Glu-Ala- | I-(Aib)-A-E-A-K | 18 |
| Lys | | |
| Ile-(Ile)-(Nme-Ala)- | I-(I)-(NMeA)-E-A-K | 38 |
| Glu-Ala-Lys | | |
| Leu-(Abu)-Ala-Glu-Ala- | L-(Abu)-A-E-A-K | 19 |
| Lys | | |
| Leu-Ile-Ala-Asn-Ala- | L-I-A-N-A-K | 10 |
| Lys | | |
| Leu-Ile-Ala-Glu-Ala- | L-I-A-E-A-A | 11 |
| Ala | | |
| Leu-Ile-Ala-Lys-Gly- | L-I-A-K-G-K | 16 |
| Lys | | |
| Leu-Pro-Ala-Glu-Ala- | L-P-A-E-A-K | 20 |
| Lys | | |
| Leu-Ile-Pro-Glu-Ala- | L-I-P-E-A-K | 21 |
| Lys | | |
| Leu-Ile-(Aib)-Glu-Ala- | L-I-(Aib)-E-A-K | 22 |
| Lys | | |
| Leu-Ile-(D-Ala)-Glu- | L-I-(D-Ala)-E-A-K | 39 |
| Ala-Lys | | |
| Leu-Ile-Ala-(D-Glu)- | L-I-A-(D-Glu)-A-K | 40 |
| Ala-Lys | | |
| Leu-Ile-Ala-(Aib)-Glu- | L-I-A-(Aib)-E-A | 23 |
| Ala | | |
| Leu-Ile-Ala-Pro-(D- | L-I-A-P-(D-Glu)-A | 41 |
| Glu)-Ala | | |
| Leu-Ile-Ala-(X.sub.1)- | L-I-A-(X₁)-A-K | 42 |
| Ala-Lys | | |
| Ile-Trp-Gly-Leu-Asp- | I-W-G-L-D-G-(bAla)-K | 24 |
| Gly-bAla-Lys | | |
| Trp-Ile-Ala-Leu-Glu- | W-I-A-L-E-G-(bAla)-K | 25 |
| Gly-bAla-Lys | | |

(Abu) = α-amino butyric acid
(Aib) = α-amino isobutyric acid
(NmeA) = N-methyl alanine
X₁ = trans-4-hydroxyproline

Similar testing to that described herein, and otherwise available to those of skill in the art to determine TGF-β-like activity, may be applied by those of skill in the art to peptides that are not included in the above analyses to determine whether or not they are TGF-β mimics.

### Example 4

### Cosmetic Treatment of Skin

A 61-year old female, of light complexion, applied a lotion comprising cytomodulin (SEQ ID NO: 1) at a concentration of about 0.001%, in EUCERIN™, to one side of her face a at a frequency of 1-2 times per week, typically at night before sleep, leaving uncovered while leaving the other side of her face without application of the TGF-β lotion. After the first treatment of topical application of the lotion, indices of skin aging, such as wrinkling (e.g., crows feet), were noticeably ameliorated on the side of the face receiving the lotion, compared to the side that did not receive lotion. For example, crows feet became more shallow and the skin and face looked fuller and smoother. Color and skin tone became more youthful in appearance. In addition, pigmentation become more even, for example, freckles and other areas of increased pigmentation (e.g., melanin) decreased in color or disappeared. After 18 days of treatment, the difference in appearance between the treated and untreated sides of the face was noticeable enough that the subject began to apply the topical composition to the entire face. No negative effects of application of the lotion were observed

The same subject applied the CM-1 lotion to the back of one hand daily, while leaving the back of the other hand untreated. The treatment continued for several weeks. After this time, freckles and other pigmented areas on the treated hand had essentially disappeared; there was no change in the appearance of the untreated hand. In addition, the skin looked plumper and thicker smoother and shinier; for example, veins appeared less prominent on the treated skin. No negative effects of the application of the lotion were observed.

### Example 5

### Cosmetic Treatment of Eyelids

A female in her 40's, of Asian ethnicity, applied a lotion comprising cytomodulin (SEQ ID NO: 1) at a concentration of about 0.001%, to her eyelids twice per week for 2 months. She reported that the effect is like having an eye lift. No adverse effects were noted.

### Example 6

### Cosmetic Treatment of Environmental and Chronological Skin Aging

A 51-year old Caucasian male, with very dry, wind-burnt skin, many fine lines, and the beginning of wrinkles, applied a lotion comprising cytomodulin (SEQ ID NO: 1) at a concentration of about 0.001% on left side of his face only, three days per week. The subject did not use any other other moisturizer of any type on his face. Within 3 weeks there was a a noticeable difference between the two sides of the face, with the right side having more fine lines appearing much drier and weather-worn than the left (treated) side. The left side looked more supple, plumper, with fewer lines. The subject stopped application after 3 months because of the difference in the appearance of the two side of the face

A 48 yr. old Caucasian female applied a lotion comprising cytomodulin (SEQ ID NO: 1) at a concentration of about 0.001% on right side of her neck only, daily. The right side in about three weeks began to look smoother, more supple, more youthful, and was smoother to the touch than the left side of the neck. The right side of the neck is noticeably different than the left side of the neck. No adverse effects were noted. The subject accidentally got the lotion in her eyes, with no apparent problem or irritation.

A 59 yr old Caucasian female with noticebly wrinkled and thickened skin on the neck applied a lotion comprising cytomodulin (SEQ ID NO: 1) at a concentration of about 0.001% to her neck once every four days. The subject also used Lancome makeup with sunscreen and Oil of Olay moisturizer for sensitive skin. The subject reports that since using the cytomodulin lotion, the wrinkles in her neck were virtually eliminated. The subject reported that there were no adverse effects.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-27. (canceled)

28. A method of cosmetic treatment of an individual comprising topically administering to an individual suffering from skin wrinkling a composition comprising a TGF-β mimic of less than about 12 amino acids in length, in a cosmetically acceptable vehicle at a concentration of about 0.0001% to about 0.01% by weight, wherein the TGF-β mimic comprises an amino acid sequence that is at least 80% identical to the amino acid sequence of SEQ ID NO: 1, and wherein, if the sequence is not identical to SEQ ID NO: 1, it contains a single amino acid substitution.

29. The method of claim 28 wherein the vehicle comprises emulsifying lanolin alcohols, waxes, and oils.

30. The method of claim 28 wherein the vehicle comprises a quaternary ammonium compound, a fatty alcohol, a fatty ester emollient, and a compound selected from the group consisting of petrolatum and mineral oil.

31. The method of claim 28 wherein the vehicle comprises water, mineral oil, isopropyl myristate, PEG-40 sorbitan peroleate, glyceryl lanolate, sorbitol, propylene glycol, cetyl palmitate, magnesium sulfate, aluminum stearate, lanolin alcohol, BHT, methylchloroisothiazolinone, and methylisothiazolinone

32. The method of claim 28 wherein the vehicle comprises water, glycerin, distearyldimonium chloride, petrolatum, isopropyl palmitate, cetyl alcohol, dimethicone, sodium chloride, methylparaben, and propylparaben.
